(19) United States
(12) Patent Application Publication
(10) Pub. No.: US 2026/0021219 A1
(43) Pub. Date: Jan. 22, 2026

(54) METHODS AND ABSORBENT ARTICLES FOR INHIBITING FECAL PROTEASE ACTIVITY

(71) Applicant: KIMBERLY-CLARK WORLDWIDE, INC., Neenah, WI (US)

(72) Inventors: Suhyoun (Su) CHON, Alpharetta, GA (US); Karien J. RODRIGUEZ, Appleton, WI (US); Penkanok SRIWIRIYANONT, Appleton, WI (US); Lindsay PEED, Loveland, OH (US)

(21) Appl. No.: 19/115,884

(22) PCT Filed: Sep. 29, 2023
(86) PCT No.: PCT/US2023/075596

§ 371 Date: Mar. 27, 2025

Related U.S. Application Data
(60) Provisional application No. 63/412,299, filed on Sep. 30, 2022.

(51) Int. Cl. A61L 15/40; A61F 13/511

(52) U.S. Cl. CPC A61L 15/40; A61F 13/51113; A61F 2013/51117; A61L 2300/434

(57) ABSTRACT

Methods and postbiotic compositions which reduce fecal enzyme activity. as well as absorbent articles which comprise the postbiotic compositions are disclosed. The postbiotic compositions and absorbent articles of the present disclosure can prevent or reduce epidermal irritation and dermatitis in diapered area by reducing the activity of fecal enzymes. In one embodiment. the postbiotic composition comprises Saccharomyces lysate derived from Saccharomyces cerevisiae.

### CROSS-REFERENCE TO RELATED APPLICATIONS

This application claims benefit of U.S. Provisional Application No. 63/412,299, filed Sep. 30, 2022, incorporated herein by reference in its entirety.

### BACKGROUND OF THE DISCLOSURE

Skin health and protection from biological insults are important for wearers of absorbent articles. Absorbent articles, such as diapers, training pants, incontinence products, and feminine care products, are often worn in direct contact with the wearer's skin. As a result, the wearer's skin can be exposed directly to various physical and biological insults, leaving the barrier function of the skin at risk.

To provide disposability, absorbent articles are often constructed of nonwoven materials. Friction between the nonwoven material and the wearer's skin is one form of physical insult to the skin barrier. Friction against the skin barrier also occurs with the use of tissue products. Tissue products are frequently used for cleansing the skin areas covered by absorbent articles (e.g., by removing biological waste materials from the skin).

In addition to these physical insults, skin covered by absorbent articles is also frequently exposed to biological insults. Biological fluids, such as urine, feces, vaginal secretions, and nasal secretions, may contain a variety of components that can damage the skin barrier. Examples of these components include proteases, lipases, and bile acids. When the skin barrier is compromised, these components, in addition to other constituents of biological fluids, can initiate or exacerbate inflammation of the skin.

Diaper dermatitis is a genre of skin conditions that, in large part, originates from impaired skin barrier function. Impairment of the skin barrier can result from a variety of factors, including increased skin hydration due to the prolonged exposure of urine or other body fluids, enzymatic skin damage due to fecal and urinary enzymes, and physical damage caused by friction against absorbent articles and excessive wiping.

Increased skin hydration disrupts skin lipid organization in the stratum corneum. This disruption increases the permeability of the skin to irritants from feces and urine, thereby increasing the risk of skin inflammation. Among these various factors, fecal enzymes are known to be a main driver for diaper rash development.

Accordingly, there is a need in the art for a topically effective composition which is capable of reducing fecal enzyme activity. By reducing fecal enzyme activity, one can effectively prevent or reduce diaper dermatitis. Furthermore, there is a need for a method of providing an absorbent article, tissue product, cream, and/or emollient that is capable of reducing fecal enzyme related activity and diaper dermatitis by mitigating exposure to fecal protease.

### SUMMARY OF THE DISCLOSURE

Disclosed herein is a method of inhibiting activity of one or more fecal enzymes, the method comprising exposing the fecal enzymes to a postbiotic composition, wherein the postbiotic composition reduces the activity of one or more of the fecal enzymes by 10% or more compared to a control.

Also disclosed herein is a method of preventing or reducing fecal enzyme-related diaper dermatitis in a subject in need thereof, the method comprising providing a postbiotic composition to the subject, wherein the postbiotic composition helps reduce or prevent fecal enzyme-related diaper dermatitis by 10% or more compared to a control.

Further disclosed herein is an absorbent article comprising a composition, wherein the composition comprises Saccharomyces lysate.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description describe examples, are explanatory, and are not restrictive of the disclosure.

### BRIEF DESCRIPTION OF THE FIGURES

The accompanying drawings, which are incorporated in and constitute a part of this specification, illustrate certain examples of the present disclosure and, together with the description, serve to explain, without limitation, the principles of the disclosure.

FIGS. 1A and 1B are graphs showing inhibition of fecal protease activity by postbiotic candidates and depict (A) % Protease inhibition in the presence of various postbiotics (maximum recommended dosages of each: 5% Oxy 229 PF, 2% Relipidium®, 2% Marsturizer™, and 2% Pauseile™, were prepared in water and added in the reaction) and (B) dose-dependent inhibition of fecal protease activity by OXY 229 PF. Protease activity of vehicle control at any given fecal protease concentration was set as 100%. 8 mM hydrochloride solution was used as a positive control for the inhibition assay;

FIG. 2 is side perspective view of an absorbent article, such as a diaper, in a fastened condition according to one implementation;

FIG. 3 is a top plan view of the absorbent article of FIG. 1 in a stretched, laid flat, unfastened condition;

FIG. 4 is a front perspective view of an absorbent article, such as a pant, according to another implementation;

FIG. 5 is a top plan view of the absorbent article of FIG. 3 in a stretched, laid flat condition; and

FIG. 6 is a front perspective cross-sectional view taken along line 5-5 from FIG. 2, with the absorbent article in a relaxed configuration.

FIG. 7 shows the effect of various S. cerevisiae extracts on fecal protease activity. Percent protease inhibition is shown in the presence of various S. cerevisiae extracts and other postbiotics. Samples were prepared at maximum recommended doses in water according to manufacturer's instruction (2% Vitacell®, 2% Sigma-Aldrich yeast extract, 2% Relipidium, and 5% OXY 229 PF) and tested for protease activity. The activity from vehicle control (water pH 7) at each given protease concentration sets as 100%. 1% lactic acid was used as positive control.

### DETAILED DESCRIPTION

### Definitions

As used herein, the singular forms “a,” “an,” and “the” include plural referents unless the context clearly dictates otherwise. Thus, for example, “a compound” includes mixtures of compounds.

As used herein, the term “about” or “approximately” means within an acceptable error range for the particular value as determined by one of ordinary skill in the art, which will depend in part on how the value is measured or determined (i.e., the limitations of the measurement system). For example, “about” can mean within 3 or more than 3 standard deviations, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, or within 5-fold, or within 2-fold, of a value.

As used herein, the terms “inhibit,” “inhibiting,” and “inhibition” mean to reduce by a measurable amount or to prevent entirely (e.g., by a decrease in activity, response, condition, disease, or another biological parameter).

As used herein, “w/v” (or “wt/vol %” or “wt/vol”) refers to the value obtained by dividing the weight of a substance (in grams) by the volume of the solution (in milliliters), and then multiplying by 100.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component (e.g., desired reduction of fecal protease). The specific level in terms of weight percentage in a composition required as an effective amount will depend upon a variety of factors, including delivery vehicle, form of composition, etc.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting to the progression of the disease or condition. This may involve only slowing the progression of the disease temporarily. The desired response to treatment of the disease or condition also can be delaying the onset or even preventing the onset of the disease or condition.

A response to a therapeutically effective dose of a disclosed composition can be measured by determining the physiological effects of the treatment or medication, such as the decrease or lack of dermatitis following administration of the composition. Other assays will be known to one of ordinary skill in the art and can be employed for measuring the level of the response. The amount of a treatment may be varied, for example, by increasing or decreasing the amount of a disclosed composition, by changing the disclosed composition administered, by changing the route of administration, by changing the timing and so on.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” or “patient” can refer to a vertebrate organism, such as a mammal (e.g., a human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as dermatitis. The effect can be therapeutic in terms of a partial or complete cure of a disease, condition, symptom or adverse effect attributed to the disease, disorder, or condition. The term “treatment” as used herein can include any treatment of fecal enzyme-related disorder or disease in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating,” can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected.

As used herein, “dose” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “postbiotic” refers to a preparation of inanimate microorganisms and/or their components that confers a health benefit on the host. Effective postbiotics contain inactivated microbial cells or cell components, with or without metabolites, that contribute to observed health benefits.

As used herein, the term “absorbent article” refers to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products (including, but not limited to, menstrual pads or pants), incontinence products and other adult care garments, medical garments, surgical pads and bandages, other personal care or health care garments, and the like without departing from the scope of the present disclosure.

As used herein, the term “tissue product” refers to products made from base webs comprising fibers and includes bath tissues, facial tissues, wet wipes, pre-moistened wipe products, cleansing and buffing pads, and other similar products. Tissue products may comprise one, two, three, or more plies. As used herein, the terms “tissue web” and “tissue sheet” refer to a fibrous sheet material suitable for forming a tissue product.

### Methods and Compositions for Inhibiting Fecal Enzyme Activity

Disclosed herein is a method of inhibiting activity of one or more fecal enzymes by exposing the fecal enzymes to a postbiotic composition. Fecal enzymes, such as serine proteases and lipases, have a direct irritant action on the skin and can cause fecal-enzyme related dermatitis. These irritant effects are increased by an increased pH and also by bile salts. A combined effect of bile salts, fecal enzymes, and increased pH can cause inflammation of the skin and can lead to fecal-enzyme related dermatitis, which is also referred to as “diaper dermatitis” or “diaper rash.” This type of rash can occur on any skin (epidermal) surface which is exposed to fecal enzymes and is not limited to diaper-wearers.

Accordingly, disclosed herein is a method of preventing or reducing fecal enzyme-related dermatitis in a subject in need thereof. The method comprises providing a postbiotic composition to the subject to inhibit activity of one or more fecal enzymes. The concept of postbiotics is based on the observation that the beneficial effects of the microbiota are mediated by the secretion of various metabolites. Postbiotics include any substance released by or produced through the metabolic activity of the microorganism, or the inactivated microorganism itself, which exerts a beneficial effect on the host, directly or indirectly. Although postbiotics do not contain live microorganisms, they show a beneficial health effect through similar mechanisms that are characteristic of probiotics.

In various implementations, the postbiotic composition disclosed herein comprises Saccharomyces lysate. For example, the Saccharomyces lysate can be derived from Saccharomyces cerevisiae.

Postbiotics can be obtained by inactivating a living microorganism. For example, thermal processing can be used, such as pasteurization, tyndallization, or autoclaving. Non-thermal inactivation techniques can also be used, such as electric field, ultrasonication, high pressure, X-rays, ionizing radiation, high-voltage electrical discharge, pulsed light, magnetic field heating, moderate magnetic field, or plasma technology. Spray drying is a method of producing a dry powder from a liquid or slurry by rapidly drying with a hot gas. Spray drying or freeze drying can be used with high inlet and/or outlet temperatures to achieve microbial inactivation. Other drying techniques, such as vacuum and fluidized bed drying, can also be used to inactivate cultures. A lysate can be obtained which contains the inactivated cells, such as Saccharomyces lysate. Examples of postbiotic preparation are known to those of skill in the art and can be found in U.S. Patent Publication No. 2022/0096571A1 and Moradi et al. (Moradi M, Molaei R, Guimarães JT. A review on preparation and chemical analysis of postbiotics from lactic acid bacteria. Enzyme Microb Technol. 2021 February; 143:109722. doi: 10.1016/j.enzmictec.2020.109722), both herein incorporated by reference in their entirety for their teachings concerning postbiotic preparation.

In addition to the postbiotic, various implementations of the composition disclosed herein can additionally include one or more amino acids. For example, the additional amino acids can include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, and/or pyrrolysine. In a specific implementation, the postbiotic composition specifically comprises one or more of valine, threonine, glutamic acid, and glycine. The postbiotic composition can further comprise disodium succinate.

In certain implementations, the postbiotic composition disclosed herein can comprise the commercial formula available as “OXY 229 PF,” which is manufactured by DSM Nutritional Products AG (DSM Nutritional Products SA/DSM Nutritional Products Ltd) of Switzerland. OXY 229 PF is a pale yellow, clear, aqueous solution for cosmetic products containing low cytoplasmic and mitochondrial constituents extracted from yeast cultures of Saccharomyces cerevisiae and amino acids. OXY 229 PF is preserved with 0.9% phenoxyethanol. Further properties of OXY 229 PF can be found in Table 2, below.

OXY 229 PF can be present in the postbiotic composition at a concentration between 0.5% and 10.0%. Specifically, OXY 229 PF can be present at 0.5%, 0.55%, 0.6%, 0.65%, 0.7%, 0.75%.0.8%,0.85%,0.9%,0.95%, 1%, 1.05%, 1.1%, 1.15%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.05%, 2.1%, 2.15%, 2.2%. 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.05%, 3.1%, 3.15%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, 4%, 4.05%, 4.1%, 4.15%, 4.2%, 4.25%, 4.3%, 4.35%, 4.4%, 4.45%, 4.5%, 4.55%, 4.6%, 4.65%, 4.7%, 4.75%, 4.8%, 4.85%, 4.9%, 4.95%, 5%, 5.05%, 5.1%, 5.15%, 5.2%, 5.25%, 5.3%, 5.35%, 5.4%, 5.45%, 5.5%, 5.55%, 5.6%, 5.65%, 5.7%, 5.75%. 5.8%, 5.85%, 5.9%, 5.95%, 6%, 6.05%, 6.1%, 6.15%, 6.2%, 6.25%, 6.3%, 6.35%, 6.4%, 6.45%, 6.5%, 6.55%, 6.6%, 6.65%, 6.7%, 6.75%, 6.8%, 6.85%, 6.9%, 6.95%. 7%, 7.05%, 7.1%, 7.15%, 7.2%, 7.25%, 7.3%, 7.35%, 7.4%, 7.45%, 7.5%, 7.55%, 7.6%, 7.65%, 7.7%, 7.75%, 7.8%, 7.85%, 7.9%, 7.95%, 8%, 8.05%, 8.1%, 8.15%, 8.2%, 8.25%, 8.3%, 8.35%, 8.4%, 8.45%, 8.5%, 8.55%, 8.6%, 8.65%, 8.7%, 8.75%, 8.8%, 8.85%, 8.9%, 8.95%, 9%, 9.05%, 9.1%, 9.15%, 9.2%, 9.25%, 9.3%, 9.35%, 9.4%, 9.45%, 9.5%, 9.55%, 9.6%, 9.65%, 9.7%, 9.75%, 9.8%, 9.85%, 9.9%, 9.95%,or 10.0% by weight, or any amount above, below, or in between these values.

According to various implementations, the postbiotic composition (e.g., OXY 229 PF) can be placed directly on the skin of the subject in need in the form of a cream, lotion, powder, or other substance. As one alternative, the postbiotic composition (e.g., OXY 229 PF) can be administered to the subject via a tissue product. As another alternative, the postbiotic composition (e.g., OXY 229 PF) can be placed directly on an absorbent article (e.g., a diaper), as described below.

The postbiotic compositions disclosed herein can reduce the activity of one or more fecal enzymes by 10% or more compared to a control. Therefore, the postbiotic compositions can reduce the activity of one or more fecal enzymes by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. For example, the amount of serine protease present in an in vitro sample or on a sample area of a subject (such as a patch of skin) can be reduced by 10% or more. In the case of an in vitro sample, fecal enzyme inhibition enzyme activities can be measured against-a-benzoyl-DL-arginine-4-nitroanilide (BAPNA) substrate (see Example 1). In an in vivo example, activity can be measured by taking a sample of the amount of serine protease present before exposure to the postbiotic composition, or it can be compared to a control with a similar starting amount of serine protease to which a postbiotic composition is not administered. One of skill in the art will readily understand how to compare results using a control.

As the postbiotic compositions herein reduce fecal enzyme activity, the postbiotic compositions disclosed herein are also reasonably expected to reduce fecal enzyme-related dermatitis by 1% or more compared to a control. Therefore, the postbiotic compositions can reduce fecal enzyme-related dermatitis by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%. 79%, 80%, 81%, 82%, 83%, 84%, 85%, 86%, 87%, 88%, 89%, 90%, 91%, 92%, 93%, 94%, 95%, 96%, 97%, 98%, 99%, or 100%. For example, the amount of fecal protease-related dermatitis present in a sample area of a subject (such as a patch of skin) can be reduced by 1% or more. This can be measured by taking a sample of the amount of serine protease present before exposure to the postbiotic composition, or it can be compared to a control with a similar starting amount of serine protease to which a postbiotic composition is not administered. One of skill in the art will readily understand how to compare results using a control.

By “reduce the activity” it is meant that the enzymatic activity of the fecal enzyme is reduced such that its ability to irritate skin, cause dermatitis, or penetrate the skin barrier is reduced compared to a control. In an in vitro setting, this can mean that the measurable activity of the fecal enzyme is reduced. In an in vivo setting, such as on the skin surface of a subject which has been exposed to a fecal enzyme, this can mean a reduction in the symptoms or severity of the symptoms associated with fecal enzyme exposure (e.g., a reduction in redness, pain, swelling, size of rash, severity of rash, or any other symptom associated with fecal enzyme-related dermatitis).

The postbiotic composition disclosed herein can also prevent fecal enzyme-related dermatitis compared to a control where the subject is not exposed to the postbiotic composition. By “prevent” it is meant that a subject with no measurable symptoms of fecal enzyme-related dermatitis prior to exposure to both the postbiotic composition and one or more fecal enzymes is prevented from developing symptoms by at least 10% compared to a control, when the control is exposed to fecal enzymes but not the postbiotic composition. Therefore, the postbiotic composition can prevent or reduce symptoms of diaper dermatitis in a subject by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. For example, a subject which shows no signs of fecal enzyme-related dermatitis can be given the postbiotic composition in the presence of fecal enzymes and continue to not show any signs of fecal enzyme-related dermatitis. This can be compared to a control, where the control receives no postbiotic composition but is exposed to a similar amount of fecal enzymes as the subject which did receive the postbiotic composition. The subject which received the postbiotic composition can have at least 10% less symptoms of diaper dermatitis than the subject which did not receive the postbiotic composition.

As discussed above, it is contemplated that the fecal enzyme can be a serine protease. Serine proteases (or serine endopeptidases) are enzymes that cleave peptide bonds in proteins. Serine serves as the nucleophilic amino acid at the enzyme's active site. They are found ubiquitously in both eukaryotes and prokaryotes. Serine proteases fall into two broad categories based on their structure: chymotrypsin-like (or trypsin-like) or subtilisin-like. Trypsin-like proteases cleave peptide bonds following a positively charged amino acid (lysine or arginine). This specificity is driven by the residue which lies at the base of the enzyme's S1 pocket (generally a negatively charged aspartic acid or glutamic acid). The S1 pocket of chymotrypsin-like enzymes is more hydrophobic than in trypsin-like proteases. This results in a specificity for medium to large sized hydrophobic residues, such as tyrosine, phenylalanine and tryptophan.

Additional components may be added to the fecal enzyme-reducing postbiotic composition of the disclosure in order to provide additional skin health benefits. For example, the postbiotic compositions of the disclosure may also include one or more emulsifying surfactants, including oil-in-water emulsifying surfactants. The surfactants provide for the incorporation of lipid (fats, oils, sterols and sterol derivatives, etc.) and other (emollient) components of the composition into the fecal enzyme-reducing postbiotic compositions. By emulsifying the lipid and other components into the composition, the surfactants contribute to the delivery of the lipids and other beneficial compounds to the skin barrier. Emulsifying surfactants are employed typically in cosmetic preparations to form emulsions of various components. The immiscible phase, such as an oil, is dispersed as droplets in the continuous phase, such as water or solvent.

Suitable surfactants include, but are not limited to, Emulsifying Wax NF, Glyceryl Stearate SE, Glycol Stearate SE, Glycereth-20 Stearate, Glyceryl Hydroxystearate, Glyceryl Laurate SE, Glyceryl Oleate SE, Propylene Glycol Oleate SE, Propylene Glycol Stearate SE, Sorbitan Stearate, water dispersible metal soaps (Sodium Stearate), Polyoxyethylene 25 Hydrogenated Castor Oil, Polyoxyethylene 75 Sorbitan Lanolin Derivative, Polyoxyethylene 50 Lanolin Derivative, Polyoxyethylene 4 Lauryl Ether, Polyoxyethylene 23 Lauryl Ether, Polyoxyethylene 10 Cetyl Ether, Polyoxyethylene 10 Stearyl Ether, Polyoxyethylene 20 Stearyl Ether, Polyoxyethylene 10 Oleyl Ether, Polyoxyethylene 20 Oleyl Ether, Polysorbate 20, Polysorbate 21, Polysorbate 40, Polysorbate 60, Polysorbate 65, Polysorbate 80, Polysorbate 81, Polysorbate 85, Dimethicone Copolymers and mixtures thereof. The surfactants of the composition may also be characterized as having a combined HLB in a range greater than 7. Therefore, one or more surfactants can be selected for use in the postbiotic composition and their combined HLB would be in a range greater than 7. The postbiotic compositions of the disclosure may include from about 1 to about 20 percent by weight of one or more emulsifying surfactants having a combined HLB in a range greater than 7. More specifically, the postbiotic compositions may include from about 2 to about 15 percent by weight of surfactants. Desirably, the postbiotic compositions of the disclosure may include from about 3 to about 10 percent by weight of surfactants. In particular aspects, the surfactants can be at least a minimum of about 1 percent by weight. The surfactants can alternatively be at least about 2, and optionally, can be at least about 3 percent by weight of the postbiotic composition. In other aspects, the surfactants can be not more than a maximum of about 20 percent by weight of the postbiotic composition. The surfactants can alternatively be not more than about 15, and optionally, can be not more than about 10 percent by weight of the postbiotic composition.

The fecal enzyme-reducing postbiotic compositions of the disclosure may also include fats and oils that provide a source of essential and non-essential fatty acids similar to those found in the skin's natural barrier. Fats and oils include compounds that are fats, oils, essential oils, fatty acids, fatty alcohols, phospholipids and mixtures of such compounds. Fats and oils include oils derived from plant and animal sources. Similarly, the essential oils include essential oils derived from plant sources. Those of skill in the art would understand that all compounds commonly understood to have the structure of or to function as fats, oils, essential oils, fatty acids, fatty alcohols and phospholipids can be used as the natural fat or oil component of the postbiotic composition of the disclosure. While an exhaustive list of each and every fat and oil that could be used in the postbiotic compositions of the disclosure is not provided, those of skill in the art will understand and appreciate the individual compounds that may serve as a fat or oil component of the postbiotic compositions of the disclosure.

Representative examples of fats and oils include, but are not limited to: Avocado Oil, Apricot Oil, Babassu Oil, Borage Oil, Camellia Oil, Canola Oil, Castor Oil, Coconut Oil, Corn Oil, Cottonseed Oil, Evening Primrose Oil, Hydrogenated Cottonseed Oil, Hydrogenated Palm Kernel Oil, Maleated Soybean Oil, Meadowfoam Oil, Palm Kernel Oil, Peanut Oil, Rapeseed Oil, Safflower Oil, Sphingolipids, Sweet Almond Oil, Tall Oil, Lanolin, Lanolin Alcohol, Lauric Acid, Palmitic Acid, Stearic Acid, Linoleic Acid, Stearyl Alcohol, Lauryl Alcohol, Myristyl Alcohol, Behenyl Alcohol, Rose Hip Oil, Calendula Oil, Chamomile Oil, Eucalyptus Oil, Juniper Oil, Sandalwood Oil, Tea Tree Oil, Sunflower Oil, and Soybean Oil. Another suitable fat/oil for the postbiotic compositions of the disclosure is PROLIPID 141 blend available from International Specialty Products of Wayne, N.J. The PROLIPID 141 blend is a mixture of glyceryl stearate, fatty acids, fatty alcohols and phospholipids.

In order to assist in replenishing skin barrier protecting and enhancing agents, the postbiotic compositions of the disclosure may include fats and oils in an amount of from about 0.1 to about 30 percent by weight, desirably from about 0.5 to about 25 percent by weight, and more desirably from about 1 to about 20 percent by weight of the postbiotic composition. In particular aspects, the fats and oils can be at least a minimum of about 0.1 percent by weight. The fats and oils can alternatively be at least about 0.5, and optionally, can be at least about 1 percent by weight of the postbiotic composition. In other aspects, the fats and oils can be not more than a maximum of about 30 percent by weight of the postbiotic composition. The fats and oils can alternatively be not more than about 25, and optionally, can be not more than about 20 percent by weight of the postbiotic composition.

The fecal enzyme-reducing postbiotic compositions of the disclosure may also include sterols and sterol derivatives that act in combination with the natural fats/oils to provide natural skin barrier enhancement and skin barrier recovery. Sterols and sterol derivatives that can be used in the postbiotic compositions of the disclosure include, but are not limited to: β-sterols having a tail on the 17 position and having no polar groups for example, cholesterol, sitosterol, stigmasterol, and ergosterol, as well as, C₁₀-C₃₀ cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyidecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd. of Parsippany, N.J.), sterol esters, and similar compounds, as well as mixtures thereof. The postbiotic compositions of the disclosure may include sterols, sterol derivatives or mixtures of both sterols and sterol derivatives in an amount of from about 0.1 to about 10 percent by weight, desirably from about 0.5 to about 5 percent by weight and more desirably from about 0.8 to about 3 percent by weight of the postbiotic composition. In particular aspects, the sterols can be at least a minimum of about 0.1 percent by weight. The sterols can alternatively be at least about 0.5, and optionally, can be at least about 0.8 percent by weight of the postbiotic composition. In other aspects, the sterols can be not more than a maximum of about 10 percent by weight of the postbiotic composition. The sterols can alternatively be not more than about 5, and optionally, can be not more than about 3 percent by weight of the postbiotic composition.

To provide improved stability and transfer to the skin of the wearer, the fecal enzyme-reducing postbiotic compositions of the disclosure may include one or more emollients. The emollients of the postbiotic compositions act as lubricants to reduce the abrasiveness of the absorbent article to the skin and, upon transfer to the skin, help to maintain the soft, smooth and pliable appearance of the skin. In general, emollients are skin-conditioning ingredients that help to soften, smooth, plasticize, lubricate, moisturize, improve the appearance of, improve the feel of and protect skin. The fecal enzyme-reducing postbiotic compositions of the disclosure may include from about 0.1 to about 10 percent by weight of one or more emollients. More specifically, the postbiotic compositions may include from about 0.5 to about 5 percent by weight of emollient(s). Even more specifically, the postbiotic compositions may include from about 1 to about 5 percent by weight of emollient(s). In particular aspects, the emollients can be at least a minimum of about 0.1 percent by weight. The emollients can alternatively be at least about 0.5, and optionally, can be at least about 1 percent by weight of the postbiotic composition. In other aspects, the emollients can be not more than a maximum of about 10 percent by weight of the postbiotic composition. The emollients can alternatively be not more than about 5 percent by weight of the postbiotic composition.

Suitable emollients include petroleum-based oils, petrolatum, vegetable oils, mineral oils, alkyl dimethicones, alkyl methicones, alkyldimethicone copolyols, phenyl silicones, alkyl trimethylsilanes, dimethicone, lanolin and its derivatives, esters, glycerol esters and their derivatives, propylene glycol esters and their derivatives, alkoxylated carboxylic acids, alkoxylated alcohols, fatty alcohols and mixtures of such compounds.

The fecal enzyme-reducing postbiotic compositions of the disclosure may also include the emollient and skin protectant, dimethicone. The dimethicone can be blended with the other components of the postbiotic composition through the addition of water-based emulsions containing dimethicone such as emulsions having the trade designations “Dow Corning 1669Emulsion” and “Dow Corning 1664 Emulsion” available from Dow Corning of Midland, Mich. The dimethicone can also be blended using a microencapsulated dimethicone such as are available from Lipo Technologies of Dayton, Ohio or from 3M of St. Paul, Minn. The dimethicone can also be added to the postbiotic compositions of the disclosure in the form of an entrapped dimethicone. Dimethicone can be entrapped in “Polytrap” or “Microsponges” as are available from Advanced Polymer Systems of San Francisco, Calif. The dimethicone can also be incorporated in the form of a dimethicone treated powder such as dimethicone-treated talc or dimethicone-treated zinc oxide as are available from KOBO of South Plainfield, N.J.

Optionally, the fecal enzyme-reducing postbiotic compositions of the disclosure may include from about 1 percent by weight to about 20 percent by weight of one or more viscosity enhancers. The viscosity enhancers can be added to increase the melt point viscosity of the postbiotic compositions. Increasing the melt point viscosity gives better stability of the postbiotic compositions on the body-facing materials of the articles. The viscosity enhancers also improve the stability of the postbiotic composition at a “hot box car” stability temperature of about 130° F. (54.5° C.). The viscosity enhancer increases the melt point viscosity of the postbiotic compositions to have a high viscosity (greater than about 50,000 centipoise) under low shear at the “hot box car” stability temperature of about 54.5° C. and at lower temperatures. Having viscosity at elevated temperatures prevents the postbiotic compositions from migrating into or away from the materials to which they are applied. However, the viscosity enhancer component also provides a low viscosity (less than about 5,000 centipoise) under shear for the postbiotic compositions at process conditions. Typically, process temperatures are approximately 5° C. above the melting point of the postbiotic composition. Generally, the process temperature is about 60° C. or higher. Different postbiotic compositions of the disclosure will have different melting points. The viscosity enhancers of the disclosure are capable of maintaining the viscosity of postbiotic compositions of the disclosure up to temperatures just below the desired processing temperature for a given composition.

Suitable viscosity enhancers can include, but are not limited to, Acrylamides Copolymers, Agar, Gelatin, Water-Dispersable Metal Soaps, Butoxy Chitosan, Calcium Carboxymethyl Cellulose, Calcium Alginate, Carbomer, Carboxybutyl Chitosan, Carboxymethyl Chitosan, Carboxymethyl Dextran, Carboxymethyl Hydroxyethyl Cellulose, Cellulose Gum, DMAPA Acrylates/Acrylic Acid/Acrylonitrogens, Hectorite, Hydrated Silica, Hydroxyethyl Cellulose, Hydroxypropyl Guar, Hydroxypropyl Methylcellulose, Isobutylene/Sodium Maleate Copolymer, Kelp, Lithium Magnesium Silicate, Lithium Magnesium Sodium Silicate, Alumina Magnesium Silicate, Smectite, Organomodified Clays, Magnesium/Aluminum/Hydroxide/Carbonate, Magnesium Aluminum Silicate, Magnesium Silicate, Magnesium Trisilicate, Methoxy PEG-22/Dodecyl Glycol Copolymer, Methyl Cellulose, Methyl Hydroxyethylcellulose, Microcrystalline Cellulose, Montmorillonite, Nonoxynol Hydroxyethylcellulose, PEG Crosspolymer, Polyacrylate-3, Polyacrylic Acid, Polyethylene/isopropyl Maleate Copolymer, Polymethacrylic Acid, Polyvinyl Alcohol, PVP/Decene Copolymer, PVP Montmorillonite, Sodium Acrylates Copolymer, Sodium Acrylate/Vinyl Alcohol Copolymer, Sodium Acrylates/Vinyl Isodecanate Crosspolymer, Partially Crosslinked Polyacrylic Acid Polymers, Sodium Carboxymethyl Starch, Sodium Hydroxypropyl Starch Phosphate, Sodium Polyacrylate, TEA Alginate, TEA Carbomer, Xanthan Gum, Locust Bean Gum, Yeast Polysaccharides and mixtures thereof.

The fecal enzyme-reducing postbiotic compositions of the disclosure may also include one or more rheology modifiers or suspending agents to prevent separation of components of the postbiotic compositions during processing. Various components of the postbiotic compositions including those that may be in particulate form or those that may be in the form of emulsion droplets are susceptible to “settling out” during the processing of the postbiotic compositions, particularly if there is an equipment shut down. The rheology modifiers of the disclosure have been found to increase the viscosity of the postbiotic compositions at process temperatures and to prevent the settling out of more dense components of the postbiotic compositions. The rheology modifiers deliver this benefit even under low shear conditions. The fecal enzyme-reducing postbiotic compositions of the disclosure may include from about 0.5 to about 10 percent by weight of a rheology modifier. Suitable rheology modifiers can be selected from natural clays, synthetic analogs of natural clays, alginates, starches, natural gums and mixtures of such compounds. Natural clays include montmorillonite, bentonite, beidellite, hectorite, saponite, stevensite, magnesium aluminum silicate and similar clays. Synthetic analogs of natural clays, such as LAPONITE synthetic clay available from Southern Clay Products, Inc. of Gonzales, Tex. can also be used to provide the rheology benefit to postbiotic compositions of the disclosure.

The fecal enzyme-reducing postbiotic compositions of the disclosure may include an emollient component. Suitable emollients that may be incorporated into the postbiotic compositions of the disclosure include oils such as petroleum based oils, petrolatum, vegetable based oils, hydrogenated vegetable oils, animal oils, hydrogenated animal oils, mineral oils, natural or synthetic oils, alkyl dimethicones, alkyl methicones, alkyldimethicone copolyols, phenyl silicones, siliconized waxes, alkyl trimethylsilanes, dimethicone, lanolin and its derivatives, esters, branched esters, glycerol esters and derivatives, propylene glycol esters and derivatives, alkoxylated carboxylic acids, alkoxylated alcohols, fatty alcohols, triglycerides, alkyl hydroxystearates and mixtures of such compounds. The esters can be selected from cetyl palmitate, stearyl palmitate, cetyl stearate, isopropyl laurate, isopropyl myristate, isopropyl palmitate, behenyl behenate, stearyl behenate, C₁₂-C₁₅ alkyl fumarate, C₂₀-C₄₀ alkyl behenate, dibehenyl fumarate, branched esters and mixtures thereof. Ethers such as eucalyptol, cetearyl glucoside, dimethyl isosorbicide polyglyceryl-3 cetyl ether, polyglyceryl-3 decyltetradecanol, propylene glycol myristyl ether and mixtures thereof can also be used as emollients. The fatty alcohols include octyldodecanol, lauryl, myristyl, cetyl, stearyl and behenyl alcohol, C₂₄ and greater fatty alcohols and mixtures thereof. For example, a particularly well-suited emollient is petrolatum. Other conventional emollients may also be added in a manner that maintains the desired properties of the postbiotic compositions set forth herein.

To provide improved stability and transfer to the skin of the wearer, the fecal enzyme-reducing postbiotic compositions of the disclosure may include from about 1 to about 95 percent by weight, desirably from about 20 to about 75 percent by weight, and more desirably from about 40 to about 60 percent by weight of the emollient. In particular aspects, the emollient can be at least a minimum of about 1 percent by weight. The emollient can alternatively be at least about 20 percent, and optionally, can be at least about 40 percent to provide improved performance. In other aspects, the emollient can be not more than a maximum of about 95 percent by weight. The emollient can alternatively be not more than about 75 percent, and optionally, can be not more than about 60 percent to provide improved effectiveness. In some instances, postbiotic compositions that include an amount of emollient greater than the recited amounts can have lower viscosities that undesirably lead to migration of the composition. Postbiotic compositions that include an amount of emollient less than the recited amounts tend to provide less transfer to the wearer's skin.

The fecal enzyme-reducing postbiotic compositions of the disclosure may also include one or more solidifying agents. The solidifying agents of the present disclosure primarily function to solidify the composition so that the postbiotic composition is a solid at room temperature and has a penetration hardness of at least 5 mm and has a melting point of at least 32° C. The solidifying agent may also provide a tackiness to the composition that improves the transfer by adhesion to the skin of the wearer. Depending on the solidifying agent selected, the solidifying agent can also modify the mode of transfer so that the postbiotic composition tends to fracture or flake off instead of actually rubbing off onto the skin of the wearer which can lead to improved transfer to the skin. The solidifying agent may further function as an emollient, occlusive agent, moisturizer, barrier enhancer, viscosity enhancer and combinations thereof. The solidifying agents may include waxes as well as compounds that perform functionally as waxes.

The solidifying agents may be selected from alkyl siloxanes, polymers, hydrogenated vegetable oils having a melting point of 35° C. or greater, fatty acid esters and branched esters with a melting point of 35° C. or greater, alkyl hydroxystearates (>C₁₆), alkoxylated alcohols and alkoxylated carboxylic acid. Additionally, the solidifying agents can be selected from animal, vegetable and mineral waxes, synthetic waxes and alkyl silicones. Examples of solidifying agents include, but are not limited to, the following: alkyl silicones, alkyl trimethylsilanes, beeswax, behenyl behenate, behenyl benzoate, C₂₄-C₂₈ alkyl dimethicone, C₃₀ alkyl dimethicone, cetyl methicone, stearyl methicone, cetyl dimethicone, stearyl dimethicone, cerotyl dimethicone, candelilla wax, carnauba, synthetic carnauba, PEG-12 carnauba, cerasin, hydrogenated microcrystalline wax, jojoba wax, microcrystalline wax, lanolin wax, ozokerite, paraffin, synthetic paraffin, cetyl esters, behenyl behenate, C₂₀-C₄₀ alkyl behenate, C₁₂-C₁₅ lactate, cetyl palmitate, stearyl palmitate, isosteryl behenate, lauryl behenate, stearyl benzoate, behenyl isostearate, cetyl myristate, cetyl octanoate, cetyl oleate, cetyl ricinoleate, cetyl stearate, decyl oleate, di-C₁₂-C₁₅ alkyl fumerate, dibehenyl fumerate, myristyl lactate, myristyl lignocerate, myristyl myristate, myristyl stearate, lauryl stearate, octyldodecyl stearate, octyldodecyl stearoyl stearate, oleyl arachidate, oleyl stearate, tridecyl behenate, tridecyl stearate, tridecyl stearoyl stearate, pentaerythrityl tetrabehenate, pentaerythritylhydrogenated rosinate, pentaerythrityl distearate, pentaerythrityl tetraabeite, pentaerythrityl tetracocoate, pentaerythrityl tetraperlargonate, pentaerythrityl tetrastearate, ethylene vinyl acetate, polyethylene, hydrogenated cottonseed oil, hydrogenated vegetable oil, hydrogenated squalene, hydrogenated coconut oil, hydrogenated jojoba oil, hydrogenated palm oil, hydrogenated palm kernel oil, hydrogenated olive oil, polyamides, metal stearates and other metal soaps, C₃₀-C₆₀ fatty alcohols, C₂₀₊ fatty amides, polypropylene, polystyrene, polybutane, polybutylene terephthalate, polydipentane, polypropylene, zinc stearate, dodecyl laurate, stearyl palmitate, octadecyl hexadecanoate, octadecyl palmitate, stearyl behenate, docosyl octanoate, tetradecyl-octadecanyl behenate, hexadecyl-cosanyl hexacosanate, shellac wax, glycol montanate, fluoranated waxes, C₂₀-C₄₀ alkyl hydroxystearyl stearate and mixtures of such compounds. Suitable branched esters include tetradecyl-octadecanyl behenate and hexadecyl-cosanyl-hexacosanate. In one aspect, the solidifying agent is a blend including about 70 weight percent cerasin wax, about 10 weight percent microcrystalline wax, about 10 weight percent paraffin wax and about 10 weight percent cetyl esters (synthetic spermaceti wax).

Appropriate solidifying agents also include alkylmethylsiloxanes that can be described as non-volatile, occlusive silicone-aliphatic hydrocarbon hybrid waxes. An example of an alkylmethylsiloxane wax is a poly(n-alkylmethylsiloxane) dimethylsiloxane. The poly(n-alkylmethylsiloxane)dimethylsiloxane can have an n-alkyl substitution of an average of 16 carbon atoms or above with an average of more than 2 alkyl groups per molecule, with hydrocarbon contents of at least 40% and with an average molecular weight of at least 1800 or higher. Examples of desirable alkylmethylsiloxanes for use in the postbiotic compositions of the disclosure include random copolymers having the following formula:
(CH₃)₃SiO((CH₃)₂SiO)ₓ(((CH₃)C₃H₆R)SiO)_{y}Si(CH₃)₃.

The “R” component of the formula can be an aliphatic hydrocarbon substituent where the chain length is from C₄ to C₄₅. In particular, “R” can be C₁₆, C₁₆₋₁₈, C₂₀₋₂₄ or C₃₀₋₄₅. For example, C₃₀₋₄₅ alkylmethylsiloxane is commercially available as trade designation “SF1642” from General Electric Silicones or “AMS-C₃₀” from Dow Corning Silicones. The value of “x” is on average more than 2 and the value of “y” is at least 1. The nature of the alkylmethylsiloxane can be balanced between its compatibility with dimethicone or polydimethyl siloxane and its compatibility with organic compounds like petrolatum and waxes. For example, as “x” increases, “y” decreases and “R” is small, the molecule increases its compatibility with dimethicone but decreases its compatibility with organic compounds. Alternatively, as “x” decreases, “y” increases and “R” is C₁₈₊, the molecule decreases its compatibility with dimethicone but increases its compatibility with organic compounds. These solidifying agents can be used to stabilize dimethicone-containing compositions of the disclosure.

To provide improved transfer to the skin of the wearer, the postbiotic composition may include from about 5 to about 95 percent by weight, desirably from about 25 to about 75 percent by weight, and more desirably from about 30 to about 50 percent by weight of solidifying agent(s). In particular aspects, the solidifying agent can be at least a minimum of about 5 percent by weight. The solidifying agent can alternatively be at least about 25 percent, and optionally, can be at least about 30 percent to provide improved performance. In other aspects, the solidifying agent can be not more than a maximum of about 95 percent by weight. The solidifying agent can alternatively be not more than about 75 percent, and optionally, can be not more than about 50 percent to provide improved effectiveness. Compositions that include an amount of solidifying agent less than the recited amounts can, in some instances, be too soft and may have lower viscosities that may undesirably lead to migration of the composition away from body-facing surfaces 11 of the absorbent article, thus diminishing transfer to the wearer's skin. Compositions that include an amount of solidifying agent greater than the recited amounts tend to provide less transfer to the wearer's skin.

The fecal enzyme-reducing postbiotic compositions of the disclosure may include one or more extracted botanical actives. The extracted botanical actives of the postbiotic compositions are extracts, containing the chemically “active” components, of various plants and plant substances. The extracted botanical actives, in combination with the other components of the postbiotic composition, provide several benefits to the skin, particularly skin that is frequently covered by an absorbent article and that is exposed to biological insults. Extracted botanical actives can include any water-soluble or oil-soluble active extracted from a particular plant. Examples of suitable extracted botanical actives are actives extracted from echinacea, yucca glauca, willow herb, basil leaves, Cuban basil, sweet basil, aspic, lavender, arkin, avocado GW, cabbage rose, Turkish oregano, carrot root, grapefruit fruit, fennel fruit, rosemary, thyme, blueberry, bell pepper, black tea, blackberry, black currant fruit, cat's claw, cemila oleifera, coffee seed, Chinese tea, dandelion root, date palm fruit, garcinia, glenn of oak, gingko leaf, green tea polyphenols (i.e. including epicatechin gallate and epigallocatechin 3-O-gallate), hawthorn berries, hexaplant richter, hibiscus special, hydrocotyl, licorice, oolong tea, sage, strawberry, sweet pea, tomato, vanilla fruit, neohesperidin, quercetin, rutin, morin, myricetin, chlorogenic acid, glutathione, glycyrrhizin, absinthe, arnica, centella asiatica, chamomile and constituents thereof (i.e., alpha-bisabolol), comfrey, cornflower, grape seed and constituents thereof (i.e., proanthocyanidins), horse chestnut, ivy (Herdera helix), magnolia, milk thistle, mimosa, oat extract, pansey, phytexcell arnica, phytoplenolin, St. John's Wort, sage GW, sage special, sedaplant richter, scullcap, seabuckthorn, white nettle, white tea, witch hazel, yarrow and any combinations thereof. Particular benefits have been observed with postbiotic compositions including echinacea, yucca glauca, willow herb, grape seed and constituents thereof (i.e., proanthocyanidins), green tea, white tea, black tea, oolong tea, Chinese tea, tea components and mixtures of such compounds. Echinacea actives may be obtained from the following echinacea species: Echinacea angustifolia, Echinacea purpurea and Echinacea pallida. Varieties of black tea include Flowery Orange Pekoe, Golden Flowery Orange Pekoe and Fine Tippy Golden Flowery Orange Pekoe. Varieties of green tea include Japanese, Green Darjeeling, apple green tea, black currant green tea, cranberry green tea, grapefruit green tea and orange green tea.

The fecal enzyme-reducing postbiotic compositions of the disclosure may include from about 0.1 to about 15 percent by weight of one or more extracted botanical actives. More specifically, the postbiotic compositions may include from about 0.5 to about 8 percent by weight of one or more extracted botanical actives. Even more specifically, the postbiotic compositions include from about 1 to about 5 percent by weight of extracted botanical actives. In particular aspects, the extracted botanical actives can be at least a minimum of about 0.1 percent by weight. The extracted botanical actives can alternatively be at least about 0.5, and optionally, can be at least about 1 percent by weight of the postbiotic composition. In other aspects, the extracted botanical actives can be not more than a maximum of about 15 percent by weight of the postbiotic composition. The extracted botanical actives can alternatively be not more than about 8, and optionally, can be not more than about 5 percent by weight of the postbiotic composition. Botanicals are primarily extracts of the plants from which they originate, and botanicals are available from suppliers as part of a composition that also contains an extracting solvent. Amounts of the botanicals in the postbiotic compositions of the disclosure in terms of active component (not extract) may range from about 0.000001 to about 15% by weight. Desirably, the amount of active botanical is from about 0.00001 to about 5% and more desirably from about 0.0001 to about 1% by weight of the postbiotic composition. Further, it is also desirable that the amount of active botanical is from about 0.0001 to about 0.5% of the postbiotic composition and more desirably from about 0.001 to about 0.1% by weight of the postbiotic composition.

The extracted botanical actives are available from numerous suppliers. For example, Echinacea extract is available from Bio-Botanica of Hauppauge, N.Y. Yucca Glauca extract is available from Brooks of South Plainfield, N.J. Canadian Willow Herb is available from Fytokem of products of Saskatchewan, Canada. Borage seed oil is available from Loders Croklaan of England. Green tea concentrate or extract (solid), green tea extract (liquid), grape seed extract (solid) and white tea 50% (solid) are available from Symrise, formerly DRAGOCO (Totowa, N.J.). The actual percentages of active botanicals in the extract composition are typically proprietary to the supplier of the extract.

If it is desired that the postbiotic compositions of the disclosure provide a treatment for the skin, the postbiotic compositions of the disclosure can also include an active ingredient such as a diaper rash skin protectant. Skin protectants are drug products that protect injured or exposed skin or mucous membrane surface from harmful or annoying stimuli. Suitable active ingredients, in addition to those mentioned above as suitable emollients, that can be incorporated into the postbiotic composition include, but are not limited to, allantoin and its derivatives, aloe, aluminum hydroxide gel, calamine, cocoa butter, dimethicone, cod liver oil, kaolin and its derivatives, lanolin and its derivatives, mineral oil, petrolatum, shark liver oil, talc, topical starch, zinc acetate, zinc carbonate, zinc oxide and mixtures thereof. The postbiotic compositions may include from about 0.10 to about 85 percent by weight of the active ingredient depending upon the skin protectant, the amount desired to be transferred to the skin and the amount required in a particular FDA skin protectant monograph.

To better enhance the benefits to the wearer, additional ingredients can be included in the fecal enzyme-reducing postbiotic compositions of the present disclosure. For example, the classes of ingredients that may be used and their corresponding benefits include, without limitation: antifoaming agents (reduce the tendency of foaming during processing); antimicrobial actives; antifungal actives; antiseptic actives; antioxidants (product integrity); antioxidants-cosmetic (reduce oxidation); astringents-cosmetic (induce a tightening or tingling sensation on skin); astringent-drug (a drug product that checks oozing, discharge, or bleeding when applied to skin or mucous membrane and works by coagulating protein); biological additives (enhance the performance or consumer appeal of the product); colorants (impart color to the product); deodorants (reduce or eliminate unpleasant odor and protect against the formation of malodor on body surfaces); other emollients (help to maintain the soft, smooth, and pliable appearance of the skin by their ability to remain on the skin surface or in the stratum corneum to act as lubricants, to reduce flaking, and to improve the skin's appearance); external analgesics (a topically applied drug that has a topical analgesic, anesthetic, or antipruritic effect by depressing cutaneous sensory receptors, or that has a topical counterirritant effect by stimulating cutaneous sensory receptors); film formers (to hold active ingredients on the skin by producing a continuous film on skin upon drying); fragrances (consumer appeal); silicones/organomodified silicones (protection, water resistance, lubricity, softness); oils (mineral, vegetable, and animal); Natural Moisturizing Factor (NMF) and other skin moisturizing ingredients known in the art; opacifiers (reduce the clarity or transparent appearance of the product); powders (enhance lubricity, oil adsorption, provide skin protection, astringency, opacity, etc.); skin conditioning agents; solvents (liquids employed to dissolve components found useful in the cosmetics or drugs); and surfactants (as cleansing agents, emulsifying agents, solubilizing agents, and suspending agents).

Before administration of the fecal enzyme-reducing postbiotic composition, the subject may have been diagnosed with fecal enzyme-related dermatitis or other fecal enzyme related disorders or diseases. For example, a physician or other caregiver can provide such a diagnosis. In another example, the subject may have been diagnosed as being susceptible to fecal enzyme-related dermatitis. In another example, the subject may use the postbiotic composition as a prophylactic or means of preventing dermatitis. The methods and compositions disclosed herein can be used as a routine part of health or can be used as needed.

The fecal enzyme-reducing postbiotic compositions disclosed herein can be administered topically. For example, the prebiotic formulation can be placed on, or integrated into, an absorbent article (discussed in more detail below). The postbiotic formulation can be placed onto or into such a product before it reaches the end user, such that the formulation is integrated into the product before it reaches the subject, or it can be placed onto the product immediately before use by the subject. When this is the case, the formulation can be placed on the substrate in the form of a spray, lotion, cream, soap, liquid, or gel, for example. In either case, the use of the absorbent article coated with the postbiotic formulation provides contact between the epidermal layer and the postbiotic composition, thereby contacts the target area with an effective amount of the composition.

The postbiotic compositions of the present disclosure may be applied to a suitable substrate, which in-turn may be used to apply the therapeutic agent to a user. Suitable applicators include a web, such as a wet laid tissue web or air laid web, gauze, cotton swab, transdermal patch, container, or holder. Particularly preferred applicators include fibrous webs, including flushable and non-flushable cellulosic webs and nonwoven webs of synthetic fibrous material. Useful webs may be wet laid, air laid, meltblown, or spunbonded. Suitable synthetic fibrous material includes meltblown polyethylene, polypropylene, copolymers of polyethylene and polypropylene, bicomponent fibers including polyethylene or polypropylene, and the like. Useful nonwoven webs may be meltblown, coform, spunbond, airlaid, hydroentangled nonwovens, spunlace, or bonded carded webs.

In certain implementations, particularly those in which the postbiotic composition is applied to a web, it may be desirable that the formulation provide certain physical attributes, such as having a smooth, lubricious, non-greasy feel; the ability to at least partially transfer from the web to the user; the capability to be retained on the web at about room temperature; or the ability to be compatible with the web manufacturing process. In certain implementations, it is preferred that at least a portion of the postbiotic composition is transferred from the tissue to the user in use.

The postbiotic composition may be applied to a web during formation of the web or after the web has been formed and dried, often referred to as off-line or post-treatment. Suitable methods of applying the postbiotic composition to a web include methods known in the art such as gravure printing, flexographic printing, spraying, WEKO™, slot die coating, or electrostatic spraying. One particularly preferred method of offline application is rotogravure printing.

### Articles of Manufacture

Also disclosed herein are various articles of manufacture which combine the fecal enzyme-reducing postbiotic composition described herein with various articles of manufacture, such as absorbent articles and/or tissue products, so as to reduce the activity of fecal enzymes. Therefore, contemplated herein is a fecal enzyme-reducing composition, such as a postbiotic composition, applied to a solid surface or impregnated into a solid matrix of any device or article of manufacture that is intended to be in contact with the epidermal area of the subject which may also be in contact with fecal enzymes. Preferably the solid surface is a flexible substrate that can be worn on or wiped on the skin or mucous membrane. In some implementations, the solid surface can be absorbent.

Accordingly, disclosed herein is an absorbent article comprising a composition, wherein the composition comprises a fecal enzyme reducing postbiotic composition, such as Saccharomyces lysate. As described above, the postbiotic composition can further comprise one or more amino acids, such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, or pyrrolysine. In a specific implementation, the postbiotic composition specifically comprises one or more of valine, threonine, glutamic acid, and glycine. The postbiotic composition can further comprise disodium succinate. In one implementation, the postbiotic composition can be OXY 229 PF, as described in detail above.

The fecal enzyme-reducing postbiotic compositions and the use of those compositions on absorbent articles and/or tissue products for inhibiting enzyme activity and subduing dermatitis of the skin have been discussed above. While the postbiotic compositions of the disclosure can have a variety of applications, the postbiotic compositions are particularly beneficial when used in conjunction with absorbent articles (e.g., diapers, incontinence garments, feminine care products, training pants, diaper pants, nursing pads and wound dressings). Additionally, the postbiotic compositions of the disclosure also provide benefits when used in conjunction with tissue products (e.g., pre-moistened wipes and cleansing and buffing pads). The postbiotic compositions disclosed herein can be disposed on a side of an absorbent article (or tissue product) configured for contact with a wearer, referred to herein as the “body-facing surface.”

When used with an absorbent article, the postbiotic composition may be applied to the entire body-facing surface of the absorbent article or may be selectively applied to particular sections of the body-facing surface (e.g., a medial section along the longitudinal centerline of the absorbent article) to facilitate transfer of the postbiotic composition to the wearer's skin. In some implementations, the body-facing surface may include multiple stripes or dots of the postbiotic composition applied thereto. In various implementations, the postbiotic composition covers a sufficient amount of the body-facing surface of the absorbent article to ensure adequate transfer to the skin. The postbiotic composition may be applied to the absorbent article in any of many well-known manners. A preferred method to uniformly apply the postbiotic composition to the body-facing surface of the absorbent article is spraying or slot coating. Spraying or slot coating the postbiotic composition is an exact process and offers control of the composition distribution and transfer rate. However, other methods, such as roto-gravure or flexographic printing and foam application can be used, as well as dip-squeeze and kiss-roll techniques. The postbiotic compositions of the present disclosure can be applied after the body-facing material has been incorporated into the absorbent article or prior to incorporating the body-facing material into the absorbent article.

The absorbent article may also include other constituents, for example, components which will improve fluid acquisition properties, fluid-wicking properties, fluid retention properties, and the like well-known in the art. Other included constituents include components which increase coherent strength (i.e., the ability to withstand deformation during use). The absorbent article may contain fibrous woven, knitted or non-woven materials, occlusive or non-occlusive films or membranes, granules, pellets or aggregates of absorbent material, synthetic polymer fibers, films, membranes and foams (e.g., nylon, polytetrafluoroethylene (PTFE, such as Teflon® or Gor-Tex®), polystyrene, polycarbonate, polyvinylchloride and polysulphone). All of these forms are well known in the art and include, for example, knitted or woven fabrics, non-woven fabrics such as felt and batting, fiber balls of cotton, rayon. cellulose or synthetic fibers, and like materials.

The fibers can be natural fibers, including but not limited to wool, silk, cotton, cellulosic fiber, and the like natural fibers. Natural polymers based on polysaccharide can also be used, including, but not limited to: modified cellulose and cellulose derivatives (e.g., alkyl-, hydroxyalkyl-, carboxymethylcellulose); gum resins (e.g., guar gum, locust bean gum, tragacanth gum, gum ontai, pectin, etc.); starch and starch derivatives (e.g., corn starch, grain starch, potato starch, amylose, amylopectin, dextrin, dextran, modified starch, hydroxy-ethyl starch, cationic starch, starch graft polymers, and the like polymers). The fibers can be synthetic fibers, including, but not limited to: polyester, polyolefin, polyamide, polyvinyl alcohol, polyvinyl acetate, polyvinyl chloride, polyvinyl urea, polyurethane, polyurea, polyacrylonitrile, as well as copolymers of these polymers, and the like synthetic fibers.

The absorbent article can be formatted into a multi-layer configuration, having an absorbent structure layer, a fluid permeable top layer which allows wicking of fluid but is itself non-wettable due to its structural composition (e.g., synthetic fiber construction), and a fluid-impermeable bottom layer (i.e., back sheet) which prevents absorbed fluid to pass from the absorbent structure layer to the adjacent tissues of the user when contacted by the absorbent article during use. Such layered configurations are well-known in the diaper and panty liner arts.

### Absorbent Articles

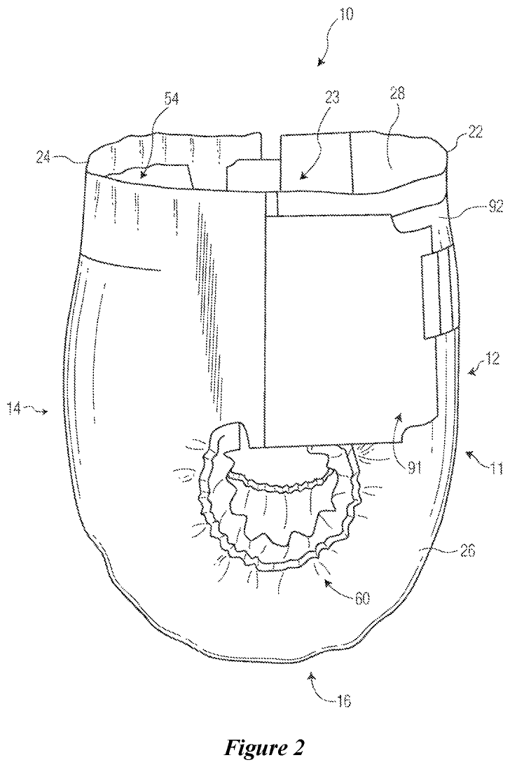

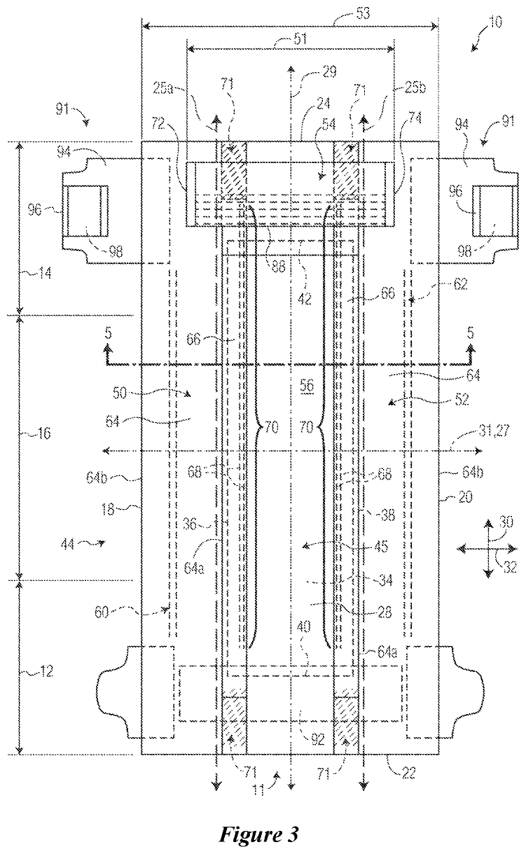

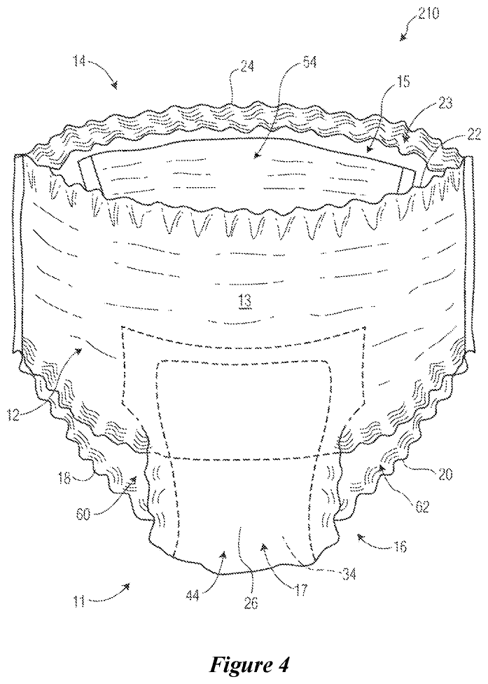

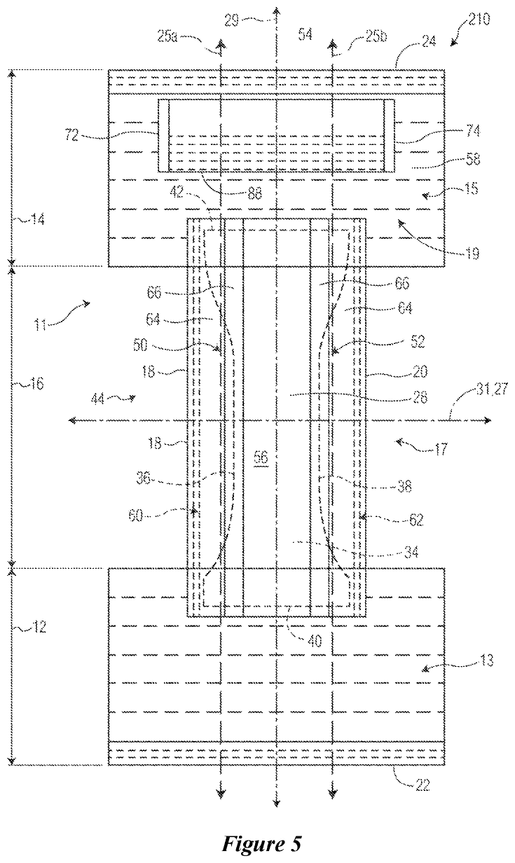

Referring to FIGS. 2 and 3, a non-limiting illustration of an absorbent article 10, for example a diaper, is illustrated. While the implementations and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross-machine direction manufacturing of a product, without departing from the spirit and scope of the disclosure. For example, the absorbent article 210 shown in FIGS. 4 and 5 provides an implementation of an absorbent article 210 that can be manufactured in crossmachine direction manufacturing process.

The absorbent article 10 illustrated in FIGS. 2 and 3 and the absorbent article 210 illustrated in FIGS. 4 and 5 can each include a chassis 11. The absorbent article 10, 210 can include a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front-end region, the rear waist region 14 can be referred to as the rear-end region, and the crotch region 16 can be referred to as the intermediate region. In the implementation depicted in FIGS. 4 and 5, a three-piece construction of an absorbent article 210 is depicted where the absorbent article 210 can have a chassis 11 including a front waist panel 13 defining the front waist region 12, a rear waist panel 15 defining the rear waist region 14, and an absorbent panel 17 defining the crotch region 16 of the absorbent article 210. The absorbent panel 17 can extend between the front waist panel 13 and the rear waist panel 15. In some implementations. the absorbent panel 17 can overlap the front waist panel 13 and the rear waist panel 15. The absorbent panel 17 can be bonded to the front waist panel 13 and the rear waist panel 15 to define a three-piece construction. However, it is contemplated that an absorbent article can be manufactured in a crossmachine direction without being a three-piece construction garment.

The absorbent article 10, 210 can have a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIGS. 2 and 3. In other implementations, the longitudinal side edges 18, 20 can be curved between the front waist edge 22 and the rear waist edge 24. In the absorbent article 210 of FIGS. 4 and 5, the longitudinal side edges 18, 20 can include portions of the front waist panel 13, the absorbent panel 17. and the rear waist panel 15.

The front waist region 12 can include the portion of the absorbent article 10, 210 that, when worn, is positioned at least in part on the front of the wearer while the rear waist region 14 can include the portion of the absorbent article 10, 210 that, when worn, is positioned at least in part on the back of the wearer. The crotch region 16 of the absorbent article 10, 210 can include the portion of the absorbent article 10, 210 that, when worn, is positioned between the legs of the wearer and can partially cover the lower torso of the wearer. The waist edges, 22 and 24, of the absorbent article 10, 210 are configured to encircle the waist of the wearer and together define a central waist opening 23 (as labeled in FIG. 2 and FIG. 4) for the waist of the wearer. Portions of the longitudinal side edges 18, 20 in the crotch region 16 can generally define leg openings for the legs of the wearer when the absorbent article 10, 210 is worn.

The absorbent article 10, 210 can include an outer cover 26 and a bodyside liner 28. The outer cover 26 and the bodyside liner 28 can form a portion of the chassis 11. In an implementation, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated implementation, can coincide with the length and width of the absorbent article 10. As illustrated in FIGS. 3 and 5, the absorbent article 10, 210 can have a longitudinal axis 29 extending in the longitudinal direction 30 and a lateral axis 31 extending in the lateral direction 32.

The bodyside liner 28 is configured to function as a body-facing surface. Accordingly, in various implementations, the bodyside liner 28 includes at least one of the postbiotic compositions described herein so as to reduce the activity of fecal enzymes. The postbiotic composition may be applied to the bodyside liner 28 or impregnated into the bodyside liner 28.

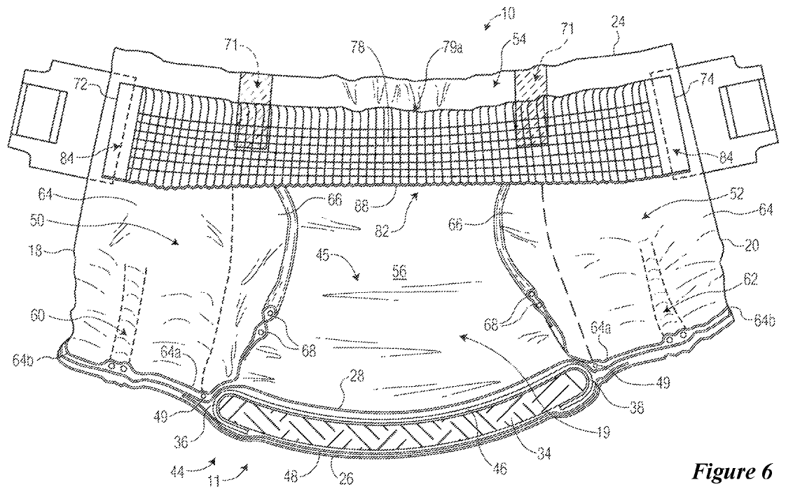

The chassis 1 I can include an absorbent body 34. The absorbent body 34 can be disposed between the outer cover 26 and the bodyside liner 28. The absorbent body 34 can have longitudinal edges, 36 and 38, which, in an implementation, can form portions of the longitudinal side edges, 18 and 20, respectively, of the absorbent article 10, 210. The absorbent body 34 can have a first end edge 40 that is opposite a second end edge 42, respectively, which, in an implementation, can form portions of the waist edges, 22 and 24, respectively, of the absorbent article 10. In some implementations, the first end edge 40 can be in the front waist region 12. In some implementations, the second end edge 42 can be in the rear waist region 14. In an implementation, the absorbent body 34 can have a length and width that are the same as or less than the length and width of the absorbent article 10, 210. The bodyside liner 28, the outer cover 26, and the absorbent body 34 can form part of an absorbent assembly 44. In the absorbent article 210 of FIGS. 4 and 5, the absorbent panel 17 can form the absorbent assembly 44. The absorbent assembly 44 can also include a fluid transfer layer 46 (as shown in FIG. 6) and a fluid acquisition layer (not shown) between the bodyside liner 28 and the fluid transfer layer 46 as is known in the art. The absorbent assembly 44 can also include a spacer layer 48 (as shown in FIG. 6) disposed between the absorbent body 34 and the outer cover 26.

The absorbent article 10, 210 can be configured to contain and/or absorb liquid, solid, and semi-solid body exudates discharged from the wearer. In some implementations, containment flaps 50, 52 can be configured to provide a barrier to the lateral flow of body exudates. To further enhance containment and/or absorption of body exudates, the absorbent article 10, 210 can suitably include a waist containment member 54. In some implementations, the waist containment member 54 can be disposed in the rear waist region 14 of the absorbent article 10, 210. Although not depicted herein, it is contemplated that the waist containment member 54 can be additionally or alternatively disposed in the front waist region 12 of the absorbent article 10, 210.

The waist containment member 54 can be disposed on the body facing surface 19 of the chassis 11 to help contain and/or absorb body exudates. In some implementations, such as in the absorbent articles 10 depicted in FIGS. 2 and 3, the waist containment member 54 can be disposed on the body facing surface 45 of the absorbent assembly 44. In some implementations, the waist containment member 54 can be disposed on the body facing surface 56 of the bodyside liner 28. In some implementations, such as in the absorbent article 210 depicted in FIGS. 4 and 5, the waist containment member 54 can be disposed on the body facing surface 58 of the rear waist panel 15.

The absorbent article 10, 210 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10, 210. The leg elastic members 60, 62 can be parallel to the longitudinal axis 29 as shown in FIGS. 3 and 5 or can be curved as is known in the art. The leg elastic members 60, 62 can be elastomeric and can provide elasticized leg cuffs.

In some implementations, the absorbent article 10, 210 can further include longitudinal extending fold lines 25a, 25b, as shown in FIGS. 3 and 5. The first longitudinal extending fold line 25a can be on one side of the longitudinal axis 29 of the absorbent article 10, 210 and the second longitudinal extending fold line 25b can be on an opposite side of the longitudinal axis 29. In some implementations, the longitudinal extending fold lines 25a, 25b can be generally parallel to the longitudinal axis 29 of the absorbent article 10, 210. In some implementations, the absorbent article 10, 210 can further include a lateral extending fold line 27. The lateral extending fold line 27 can be parallel to and located at the lateral axis 31 of the absorbent article 10, 210 in some implementations.

Additional details regarding each of these elements of the absorbent article 10, 210 described herein can be found below and with reference to the Figures.

Outer cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an implementation, for example, the outer cover 26 can be constructed of a microporous polymeric film, such as polyethylene or polypropylene.

In an implementation, the outer cover 26 can be a single layer of a liquid impermeable material, such as a polymeric film. In an implementation, the outer cover 26 can be suitably stretchable, and more suitably elastic, in at least the lateral direction 32 of the absorbent article 10, 210. In an implementation, the outer cover 26 can be stretchable, and more suitably elastic, in both the lateral 32 and the longitudinal 30 directions. In an implementation, the outer cover 26 can be a multi-layered laminate in which at least one of the layers is liquid impermeable. In some implementations, the outer cover 26 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A. G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 210 and wetting articles, such as bed sheets and clothing. as well as the wearer and caregiver.

In some implementations, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

Bodyside liner:

The bodyside liner 28 of the absorbent article 10. 110, 210 can overlay the absorbent body 34 and the outer cover 26 and can isolate the wearer's skin from liquid waste retained by the absorbent body 34. In various implementations, a fluid transfer layer 46 can be positioned between the bodyside liner 28 and the absorbent body 34. In various implementations, an acquisition layer (not shown) can be positioned between the bodyside liner 28 and the absorbent body 34 or a fluid transfer layer 46, if present. In various implementations, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an implementation, the bodyside liner 28 can extend beyond the absorbent body 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. The bodyside liner 28 may be narrower than the outer cover 26. However, in other implementations, the bodyside liner 28 and the outer cover 26 may be of the same dimensions in width and length. In other implementations, the bodyside liner 28 can be of greater width than the outer cover 26. It is also contemplated that the bodyside liner 28 may not extend beyond the absorbent body 34 and/or may not be secured to the outer cover 26. In some implementations, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 to Kirby, Scott S.C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an implementation, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an implementation, a bodyside liner 28 can be a spunbond substrate with a basis weight from 10 or 12 to 15 or 20 gsm. In an implementation, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an implementation, the bodyside liner 28 can be stretchable, and more suitably elastic. In an implementation, the bodyside liner 28 can be suitably stretchable and more suitably elastic in at least the lateral or circumferential direction of the absorbent article 10, 210. In other aspects, the bodyside liner 28 can be stretchable, and more suitably elastic, in both the lateral and the longitudinal directions 32, 30, respectively.

In various implementations, the bodyside liner 28 includes a composition comprising a fecal enzyme reducing postbiotic composition, such as Saccharomyces lysate. As described above, the postbiotic composition can further comprise one or more amino acids, such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, or pyrrolysine. In a specific implementation, the postbiotic composition specifically comprises one or more of valine, threonine, glutamic acid, and glycine. The postbiotic composition can further comprise disodium succinate. In one implementation, the postbiotic composition can be OXY 229 PF, as described in detail above.

The fecal enzyme-reducing postbiotic compositions and the use of those compositions on absorbent articles for inhibiting enzyme activity and subduing dermatitis of the skin have been discussed above. In various implementations, the postbiotic composition may be applied to the entire bodyside liner 28 or may be selectively applied to particular sections of the bodyside liner 28 (e.g., a medial section along the longitudinal centerline of the bodyside liner 28) to facilitate transfer of the postbiotic composition to the wearer's skin. In some implementations, the bodyside liner 28 may include multiple stripes or dots of the postbiotic composition applied thereto. In various implementations, the postbiotic composition covers a sufficient amount of the bodyside liner 28 to ensure adequate transfer to the wearer's skin.

The postbiotic composition may be applied to the bodyside liner 28 in any of many well-known manners. A preferred method to uniformly apply the postbiotic composition to the bodyside liner 28 is spraying or slot coating. Spraying or slot coating the postbiotic composition is an exact process and offers control of the composition distribution and transfer rate. However, other methods, such as roto-gravure or flexographic printing and foam application can be used, as well as dip-squeeze and kiss-roll techniques. The postbiotic compositions of the present disclosure can be applied after the bodyside liner 28 has been incorporated into the absorbent article 10, 210 or prior to incorporating the bodyside liner 28 into the absorbent article 10, 210.

### Containment Flaps:

In an implementation, the absorbent article 10, 210 can include a pair of containment flaps 50, 52. The containment flaps 50, 52 can be formed separately from the absorbent chassis 11 and attached to the chassis 11 or can be formed integral to the chassis 11. In some implementations, the containment flaps 50, 52 can be secured to the chassis 11 of the absorbent article 10, 210 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an implementation, the containment flaps 50, 52 can extend generally in a longitudinal direction 30 from the front waist region 12 of the absorbent article 10, through the crotch region 16 to the rear waist region 14 of the absorbent article 10. In some implementations, the containment flaps 50, 52 can extend in a direction substantially parallel to the longitudinal axis 29 of the absorbent article 10, 210, however, in other implementations, the containment flaps 50, 52 can be curved, as is known in the art. In other implementations, such as the absorbent article 210 in FIGS. 4 and 5, the containment flaps 50, 52 can be disposed on the absorbent panel 17 in the crotch region 16.

In implementations where the containment flaps 50, 52 are coupled to the chassis 11, the containment flaps 50, 52 can be bonded to the bodyside liner 28 with a barrier adhesive 49, as shown in FIG. 6. Alternatively, the containment flaps 50, 52 can be bonded to the outer cover 26 with a barrier adhesive 49, or to the spacer layer 48. Of course, the containment flaps 50, 52 can be bonded to other components of the chassis 11 and can be bonded with other suitable means other than a barrier adhesive 49. The containment flaps 50, 52 can be constructed of a fibrous material which can be similar to the material forming the bodyside liner 28. Other conventional materials, such as polymer films, can also be employed.

The containment flaps 50, 52 can each include a base portion 64 and a projection portion 66. The base portion 64 can be bonded to the chassis 11, for example, to the bodyside liner 28 or the outer cover 26 as mentioned above. The base portion 64 can include a proximal end 64a and a distal end 64b. The projection portion 66 can be separated from the base portion 64 at the proximal end 64a of the base portion 64. As used in this context, the projection portion 66 is separated from the base portion 64 at the proximal end 64a of the base portion 64 in that the proximal end 64a of the base portion 64 defines a transition between the projection portion 66 and the base portion 64. The proximal end 64a of the base portion 64 can be located near the barrier adhesive 49. In some implementations, the distal ends 64b of the base portion 64 can laterally extend to the respective longitudinal side edges 18, 20 of the absorbent article 10, 210. In other implementations, the distal ends 64b of the base portion 64 can end laterally inward of the respective longitudinal side edges 18, 20 of the absorbent article 10, 210. The containment flaps 50, 52 can also each include a projection portion 66 that is configured to extend away from the body facing surface 19 of the chassis 11 at least in the crotch region 16 when the absorbent article 10, 210 is in a relaxed configuration, as illustrated in FIG. 6. The containment flaps 50, 52 can include a tack-down region 71 in either or both of the front waist region 12 and the rear waist region 14 where the projection portion 66 is coupled to the body facing surface 19 of the chassis 11.

It is contemplated that the containment flaps 50, 52 can be of various configurations and shapes, and can be constructed by various methods. For example, the containment flaps 50, 52 of FIG. 6 depict a vertical containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10, 210. However, the containment flaps 50, 52 can include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 210 and still remain within the scope of this disclosure.

The ontainnment flaps 50, 52 can include one or more flap elastic members 68, such as the two flap elastic strands depicted in FIG. 6. Suitable elastomeric materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastomeric properties itself.

The flap elastic members 68, as illustrated in FIG. 6, can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, 210, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, 110% to 350%. In one implementation, the elastic members 68 can be coated with adhesive while elongated to a specified length prior to attaching to the elastic members 68 to the containment flaps 50, 52. In a stretched condition, the length of the elastic members 68 which have adhesive coupled thereto can provide an active flap elastic region 70 in the containment flaps 50, 52, as labeled in FIG. 3, which will gather upon relaxation of the absorbent article 10. The active flap elastic region 70 of containment flaps 50, 52 can be of a longitudinal length that is less than the length of the absorbent article 10, 210. In this example method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 in the active flap elastic region 70 when the absorbent article 10, 210 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28), as depicted in FIG. 6.

Of course, the elastic members 68 can be bonded to the containment flaps 50, 52 in various other ways as known by those of skill in the art to provide an active flap elastic region 70, which is within the scope of this disclosure. Additionally, the active flap elastic regions 70 can be shorter or longer than depicted herein, including extending to the front waist edge 22 and the rear waist edge 24, and still be within the scope of this disclosure.

### Leg Elastics:

Leg elastic members 60, 62 can be secured to the outer cover 26, such as by being bonded thereto by laminate adhesive, generally laterally inward of the longitudinal side edges, 18 and 20, of the absorbent article 10, 210. The leg elastic members 60, 62 can form elasticized leg cuffs that further help to contain body exudates. In an implementation, the leg elastic members 60, 62 may be disposed between inner and outer layers (not shown) of the outer cover 26 or between other layers of the absorbent article 10, for example, between the base portion 64 of each containment flap 50, 52 and the bodyside liner 28 as depicted in FIG. 6, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastomeric materials may be used for the leg elastic members 60, 62.

Suitable elastomeric materials can include sheets, strands or ribbons of natural rubber, synthetic rubber, or thermoplastic elastomeric materials. The elastomeric materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some implementations. Of course, the leg elastic members 60, 62 can be omitted from the absorbent article 10, 210 without departing from the scope of this disclosure.

### Waist Containment Member:

In an implementation, the absorbent article 10, 210 can have one or more waist containment members 54. The waist containment member(s) 54 can be disposed in the rear waist region 14 as illustrated in FIGS. 2-6. In general, the waist containment member 54 can help contain and/or absorb body exudates, especially low viscosity fecal matter, and as such, can be preferred to be in the rear waist region 14. In some implementations, the absorbent article 10, 210 can have a waist containment member 54 disposed in the front waist region 12. A waist containment member 54 in the front waist region 12 can help contain and/or absorb body exudates, such as urine, in the front waist region 12. Although not as prevalent as in the rear waist region 14, in some circumstances, fecal material may also spread to the front waist region 12, and thus, a waist containment member 54 disposed in the front waist region 12 can help contain and/or absorb body exudates as well. In other implementations, the absorbent article 10, 210 can have a waist containment member 54 in both the rear waist region 14 and the front waist region 12.

The waist containment member 54 can be disposed on the body facing surface 45 of the absorbent assembly 44. In some implementations, such as in implementations illustrated in FIGS. 2-3 and 6, the waist containment member 54 can be disposed on the body facing surface 56 of the bodyside liner 28. However, in some implementations, such as the absorbent article 210 in FIG. 5. the waist containment member 54 can be disposed on a body facing surface 58 of the rear waist panel 15.

The waist containment member 54 can include a first longitudinal side edge 72 and a second longitudinal side edge 74. The first longitudinal side edge 72 can be opposite from the second longitudinal side edge 74. The distance between the first longitudinal side edge 72 and the second longitudinal side edge 74 can define a width 51 of the waist containment member 54 in the lateral direction 32, as shown in FIG. 3.

As illustrated in FIGS. 3 and 6, the waist containment member 54 can be configured such that the first longitudinal side edge 72 can be disposed laterally outward of the proximal end 64a of the base portion 64 of the containment flap 50. Similarly, the waist containment member 54 can be configured such that the second longitudinal side edge 74 can be disposed laterally outward of the proximal end 64a of the base portion 64 of the containment flap 52. The waist containment member 54 can be configured such that the width 51 of the waist containment member 54 can be greater than a lateral distance between longitudinal extending fold lines 25a, 25b, as shown in FIGS. 3 and 5.

The waist containment member 54 can also include a proximal portion (not shown) and a distal portion 78. The proximal portion can be coupled to the body facing surface 19 of chassis 11 (e.g., the body facing surface 45 of the absorbent assembly 44 or the body facing surface 56 of the bodyside liner 28) whereas the distal portion 78 of the waist containment member 54 can be free to move with respect to the chassis 11 and the absorbent assembly 44 when the absorbent article 10, 210 is in the relaxed configuration, such as shown in FIG. 6. When the waist containment member 54 is in a relaxed configuration, the distal portion 78 extends away from the chassis 11 and absorbent assembly 44 in a vertical direction, which is perpendicular to the plane defined by the longitudinal axis 29 and the lateral axis 31. A fold 79a can separate the proximal portion from the distal portion 78 of the waist containment member 54. As used in this context, the fold 79a separates the proximal portion from the distal portion 78 in that the fold 79a defines a transition between the proximal portion and the distal portion 78.

In some implementations, the proximal portion of the waist containment member 54 can be coupled to the body facing surface 56 of the bodyside liner 28. In other implementations, the proximal portion of the waist containment member 54 can be coupled to the body facing surface 58 of the rear waist panel 15. The proximal portion can be coupled to the body facing surface 45 by an adhesive, by pressure bonding, by ultrasonic bonding, by thermal bonding, and combinations thereof.

Because the distal portion 78 of the waist containment member 54 can freely move with respect to the absorbent assembly 44 when the absorbent article 10, 210 is in the relaxed configuration, the distal portion 78 can help provide a containment pocket 82 when the absorbent article 10, 210 is in the relaxed configuration. The containment pocket 82 can help provide a barrier to contain and/or can help absorb body exudates. The containment pocket 82 can be especially beneficial for containing and/or absorbing low viscosity fecal matter, which can be prevalent in younger children. The first longitudinal side edge 72 can be disposed laterally outward of the proximal end 64a of the base portion 64 of the containment flap 50, and thus, the containment pocket 82 can extend laterally outward of the proximal end 64a of the containment flap 50. Similarly, the second longitudinal side edge 74 can be disposed laterally outward of the proximal end 64a of the base portion 64 of the containment flap 52 and the containment pocket 82 can extend laterally outward of the proximal end 64a of the containment flap 52. Such a configuration provides waist containment member 54 with a wide containment pocket 82 to contain and/or absorb body exudates.

To help prevent lateral flow of body exudates that are contained by the containment pocket 82 of the waist containment member 54, the distal portion 78 of the waist containment member 54 can be bonded to the proximal portion of the waist containment member 54 and/or the body facing surface 19 of the chassis 11 near the first and second longitudinal side edges 72, 74, respectively. For example, FIG. 6 depicts tack-down regions 84 where the distal portion 78 of the waist containment member 54 can be bonded to the proximal portion of the waist containment member 54 and/or the body facing surface 19 of the chassis 11.

In preferred implementations, the waist containment member 54 can include at least one elastic member and even more elastic members in further implementations. Generally, the elastic member can span substantially from the first longitudinal side edge 72 to the second longitudinal side edge 74 of the waist containment member 54. The elastic member can be disposed in the distal portion 78 of the waist containment member 54, and preferably, is located near a free edge 88 of the distal portion 78 of the waist containment member 54.

A wide variety of elastomeric materials may be used for the elastic member(s) in the waist containment member 54. Suitable elastomeric materials can include sheets, strands or ribbons of natural rubber, synthetic rubber, elastic foams, or thermoplastic elastomeric materials (e.g., films). The elastomeric materials can be stretched and secured to a substrate forming the waist containment member 54, secured to a gathered substrate, or secured to a substrate and then elasticized or shrunk, for example, with the application of heat, such that the elastic retractive forces are imparted to the substrate forming the waist containment member 54.

The waist containment member 54 can be disposed to be coupled to the chassis 11 by being placed either over the containment flaps 50, 52 or under the containment flaps 50, 52. More specifically, the waist containment member 54 can be disposed on the body facing surface 19 of the chassis 11 such that the proximal portion of the waist containment member 54 is disposed over the base portion 64 of the first and the second containment flaps 50, 52, respectively. Alternatively, the waist containment member 54 can be disposed on the body facing surface 19 of the chassis 11 such that the proximal portion of the waist containment member 54 is disposed under the base portion 64 of the first and the second containment flaps 50, 52, respectively. Both configurations can provide advantages to the functioning of the waist containment member 54 to contain and/or absorb body exudates.

Where the proximal portion of the waist containment member 54 is disposed over the base portion 64 of the containment flaps 50, 52, the containment flaps 50, 52 can have an active flap elastic region 70 that longitudinally overlaps with the distal portion 78 of the waist containment member 54 when the absorbent article 10 is in the stretched, laid flat configuration, such as illustrated in FIG. 3. Additionally or alternatively, the tack-down region 71 may not extend from the rear waist edge 24 to the free edge 88 of the distal portion 78 of the waist containment member 54, such as illustrated in FIG. 3.

Where the proximal portion of the waist containment member 54 is disposed under the base portion 64 of the containment flaps 50, 52, the tack-down region 71 of the projection portion 66 of each of the containment flaps 50, 52 may longitudinally overlap with the distal portion 78 of the waist containment member 54. In some of these implementations, the tack-down region 71 of projection portion 66 of each of the containment flaps 50, 52 can extend to the free edge 88 of the waist containment member 54 to further assist in containing exudates to the containment pocket 82 created by the waist containment member 54.

The waist containment member 54 can be comprised of a variety of materials. In a preferred implementation, the waist containment member 54 can be comprised of a spunbond-meltblown-spunbond (“SMS”) material. However, it is contemplated that the waist containment member 54 can be comprised of other materials including, but not limited to, a spunbond-film-spunbond (“SFS”), a bonded carded web (“BOW”), or any non-woven material. In some implementations, the waist containment member 54 can be comprised of a laminate of more than one of these example materials, or other materials. In some implementations, the waist containment member 54 can be comprised of a liquid impermeable material. In some implementations, the waist containment member 54 can be comprised of a material coated with a hydrophobic coating. The basis weight of the material forming the waist containment member 54 can vary, however, in a preferred implementation, the basis weight can be between 8 gsm to 120 gsm, not including the elastic members 86 in the waist containment member 54. More preferably, the basis weight of the material comprising the waist containment member 54 can be between 10 gsm to 40 gsm, and even more preferably, between 15 gsm to 25 gsm.

### Fastening System:

In an implementation, the absorbent article 10 can include a fastening system. The fastening system can include one or more back fasteners 91 and one or more front fasteners 92. The implementations shown in FIGS. 2 and 3 depict implementations with one front fastener 92. Portions of the fastening system may be included in the front waist region 12, rear waist region 14, or both.

The fastening system can be configured to secure the absorbent article 10 the waist of the wearer in a fastened condition as shown in FIG. 2 and help maintain the absorbent article 10 in place during use. In an implementation, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 3. As shown in FIG. 6, in some implementations the waist containment member 54 can extend to back fasteners 91. In some implementations, the waist containment member 54 can be coupled to the stretch component 94 of the back fasteners 91, either directly or indirectly. In some implementations, the waist containment member 54 can extend to the longitudinal side edges 18, 20 of the absorbent article 10, 210.

Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, 1-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10, 210. The absorbent body 34 can have a length and width that can be less than or equal to the length and width of the absorbent article 10, 210.

In an implementation, the absorbent body 34 can be composed of absorbent material, such as fibrous absorbent material and/or, superabsorbent material, binder materials, surfactants, selected hydrophobic and hydrophilic materials, pigments, lotions, odor control agents or the like, as well as combinations thereof. In an implementation, the absorbent body 34 can be a matrix of cellulosic fluff and superabsorbent material. In another implementation, the absorbent material of the absorbent body 34 can comprise only superabsorbent material. In an implementation, the absorbent body 34 may be constructed of a single layer of materials, or in the alternative, may be constructed of two or more layers of materials.

When composed at least partially of fibrous material, various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber.

When composed at least partially of superabsorbent materials, such superabsorbent materials can be selected from natural, synthetic, and modified natural polymers and materials. The superabsorbent materials can be inorganic materials, such as silica gels, or organic compounds, such as cross-linked polymers.

If a spacer layer 48 is present, the absorbent body 34 can be disposed on the spacer layer 48 and superposed over the outer cover 26. The spacer layer 48 can be bonded to the outer cover 26, for example, by adhesive. In some implementations, a spacer layer 48 may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an implementation. the outer cover 26 can be composed of a single layer and the absorbent body 34 can be in contact with the singer layer of the outer cover 26. In some implementations, at least a portion of a layer, such as but not limited to, a fluid transfer layer 46 and/or a spacer layer 48, can be positioned between the absorbent body 34 and the outer cover 26, such as illustrated in FIG. 6. The absorbent body 34 can be bonded to the fluid transfer layer 46 and/or the spacer layer 48.

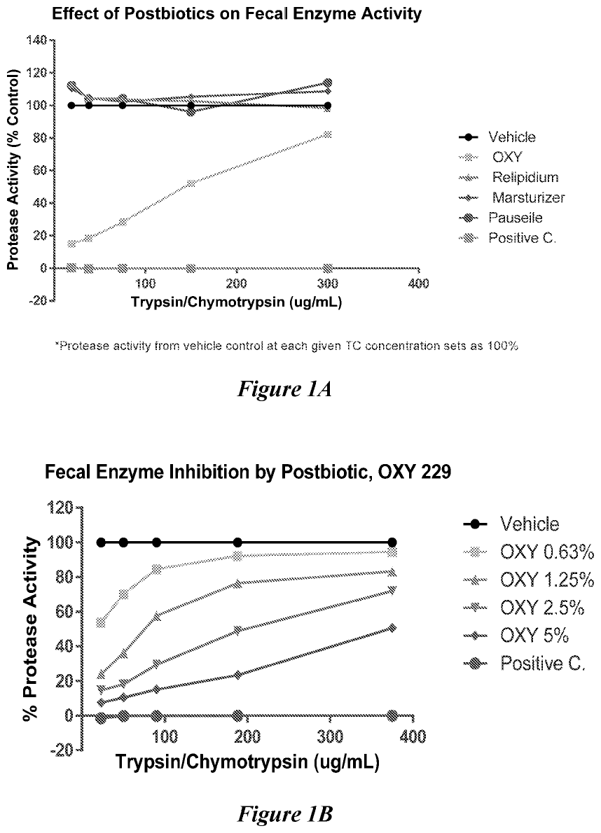

Although the FIGS. 1-5 focus on description of a diaper absorbent article 10, 210, it should be understood that the absorbent structures of the present disclosure (e.g., the absorbent body 34) may be used in any absorbent article—including but not limited to diapers, diaper pants, training pants, youth pants, swim pants, feminine hygiene products, including, but not limited to, menstrual pads or pants, incontinence products and other adult care garments, medical garments, surgical pads and bandages, other personal care or health care garments, and the like.

### Tissue Product

The postbiotic compositions of the present disclosure may also be used in combination with a tissue product. As discussed above, tissue products are made from base webs comprising fibers and include bath tissues, facial tissues, wet wipes, pre-moistened wipe products, cleansing and buffing pads, and other similar products. Tissue products may comprise one, two, three, or more plies. As used herein, the terms “tissue web” and “tissue sheet” refer to a fibrous sheet material suitable for forming a tissue product.

In various implementations, the postbiotic composition may be incorporated into or onto a tissue web so as to reduce the activity of fecal enzymes. Therefore, contemplated herein is a fecal enzyme-reducing composition, such as a postbiotic composition, applied to a tissue web or impregnated into a tissue web that is intended to be in contact with the epidermal area of the subject which may also be in contact with fecal enzymes.

In various implementations, the tissue product includes a composition comprising a fecal enzyme reducing postbiotic composition, such as Saccharomyces lysate. As described above, the postbiotic composition can further comprise one or more amino acids, such as alanine. arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, or pyrrolysine. In a specific implementation, the postbiotic composition specifically comprises one or more of valine, threonine, glutamic acid. and glycine. The postbiotic composition can further comprise disodium succinate. In one implementation, the postbiotic composition can be OXY 229 PF, as described in detail above.

The fecal enzyme-reducing postbiotic compositions and the use of those compositions on tissue products for inhibiting enzyme activity and subduing dermatitis of the skin have been discussed above. In various implementations, the postbiotic composition may be applied to the entire tissue product or may be selectively applied to particular sections of the tissue product to facilitate transfer of the postbiotic composition to the wearer's skin. In various implementations, the postbiotic composition covers a sufficient amount of the tissue product to ensure adequate transfer to the wearer's skin.

In various implementations, the postbiotic composition may be used in combination with a tissue web to form a wet wipe (or may be included in a wetting composition for use in combination with a wipe which may be dispersible). In other implementations, the postbiotic composition may be incorporated into wipes such as wet wipes, hand wipes, face wipes, cosmetic wipes, cloths, and the like.

In one implementation, the wet wipe may comprise a nonwoven material that is wetted with an aqueous solution (a wetting composition), which may include (or be composed entirely of) at least one of the postbiotic compositions disclosed herein. As used herein, the nonwoven material comprises a fibrous material or substrate, where the fibrous material or substrate comprises a sheet that has a structure of individual fibers or filaments randomly arranged in a mat-like fashion. Nonwoven materials may be made from a variety of processes including, but not limited to, airlaid processes, wet-laid processes such as with cellulosic-based tissues or towels, hydroentangling processes, staple fiber carding and bonding, melt blown, and solution spinning.

The fibers forming the fibrous material may be made from a variety of materials including natural fibers, synthetic fibers, and combinations thereof. The choice of fibers may depend upon, for example, the intended end use of the finished substrate and the fiber cost. For instance, suitable fibers may include, but are not limited to, natural fibers such as cotton, linen, jute, hemp, wool, wood pulp, etc. Similarly, suitable fibers may also include: regenerated cellulosic fibers, such as viscose rayon and cuprammonium rayon; modified cellulosic fibers, such as cellulose acetate; or synthetic fibers, such as those derived from polypropylenes, polyethylenes, polyolefins, polyesters, polyamides, polyacrylics, etc. Regenerated cellulose fibers, as briefly discussed above, include rayon in all its varieties as well as other fibers derived from viscose or chemically modified cellulose, including regenerated cellulose and solvent-spun cellulose, such as Lyocell. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Chemically treated natural cellulosic fibers may be used, such as mercerized pulps, chemically stiffened or crosslinked fibers, or sulfonated fibers.

In addition, cellulose produced by microbes and other cellulosic derivatives may be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. Blends of one or more of any of the previously described fibers may also be used, if so desired.

The fibrous material may be formed from a single layer or multiple layers. In the case of multiple layers, the layers are generally positioned in a juxtaposed or surface-to-surface relationship and all or a portion of the layers may be bound to adjacent layers. The fibrous material may also be formed from a plurality of separate fibrous materials wherein each of the separate fibrous materials may be formed from a different type of fiber.

Airlaid nonwoven fabrics are particularly well suited for use as wet wipes. The basis weights for airlaid nonwoven fabrics may range from about 20 to about 200 grams per square meter (gsm) with staple fibers having a denier of about 0.5 to about 10 and a length of about 6 to about 15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

Processes for producing airlaid non-woven basesheets are described in, for example, published U.S. Pat. App. No. 2006/0008621, herein incorporated by reference to the extent it is consistent herewith.

Unless defined otherwise, all scientific and technical terms used herein have the same meaning as commonly understood by those skilled in the relevant art. The Examples of implementations are for illustration only.

### EXAMPLES

To further illustrate the principles of the present disclosure, the following examples are put forth to provide those of ordinary skill in the art with a description of how the compositions, articles, and methods claimed herein are made and evaluated. They are intended to be examples and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Inhibition of Fecal Enzymes by Postbiotic Formulation, OXY 229 PF (Saccharomyces Cerevisiae Lysate)

Five postbiotic candidates were identified based on their skin health potential and commercial availability. Detailed information is in Table 1, below.

Since fecal enzymes are the primary rash-causing skin irritants in diapered areas, the ability of five postbiotic candidates to inhibit fecal protease activity was evaluated in vitro. The detailed protocol of enzyme activity assay is below.

### Fecal Enzyme Inhibition Assay

BAPNA (Na-Benzoyl-DL-Arginine-p-Nitroanilide) assay was performed to evaluate whether postbiotic candidates possess inhibitory action for fecal proteases (chymotrypsin/trypsin). Postbiotic samples were prepared in a vehicle (water, pH 7) at the maximum recommended dose of each candidate based on Table. 1.25 μl of the prepared solution (5% OXY 229 PF, 2% Relipid®, 2% Marsturizer™, or 2% Pauseile™) was mixed with various dosages of 25 μl of fecal protease mix (trypsin and chymotrypsin Specialty enzyme). Finally, 50 μl of 5 mM BAPNA substrate was added right before the assay and then measured optical density (OD) @ 405 nm for 10 minutes. Protease activity was calculated by measuring the p-NA (p-Nitroanilide) released from the substrate based on the standard curve. The degree of inhibition on fecal protease activity was expressed as % vehicle control. Results of the enzyme activity assay are as below.

### OXY229 PF (Saccharomyces Cerevisiae Lysate) Inhibited Fecal Enzyme Activity In A Dose-Dependent Manner

The percent reduction of protease activity was monitored with or without postbiotic in the reaction mixture at various concentrations of fecal protease. SymReboot® was excluded from the testing since it created a turbid solution, interfering with the assay. Among four tested at their maximum recommended dose, OXY 229 PF decreased fecal protease activity when 23 to 375mg/mL of enzymes were present in the reaction (FIG. 1A). Inhibition of fecal protease activity by OXY 229 PF was further confirmed in the reaction with serial dilution of OXY 229 PF. Dose-dependent inhibition was observed in every concentration of fecal protease tested, clearly demonstrating its capability of reducing fecal enzyme activity (FIG. 1B). OXY 229 PF stock solution contains glycerol up to 25%. There was no inhibition mediated by glycerol. Other postbiotics tested were in a similar pH range, indicating that pH was not a factor in mechanism of action. Only OXY 229 PF showed fecal enzyme inhibition among others (FIG. 1A).

OXY 229 PF is a skin care ingredient containing postbiotics (Saccharomyces cerevisiae lysate) and other actives such as glycerol and amino acids (Valine, Threonine, Glutamic acid, Glycine, and Disodium Succinate). It is manufactured by DSM, which markets it for cosmetic applications on the face, stating that it is, “A skin bioactive ingredient designed to revive dull looking skin.” They also indicate that its uses in skin health comprise 1) a mitochondrial respiration activator; 2) ability to boost oxygen uptake by up to 750%; 3) ability to increase cell viability by up to 10%; 4) ability to increase cell turnover activity by up to 25%; and 5) reduction of sebum and Corynebacterium kroppenstedtii levels-a target to control skin redness. There has been no report that OXY 229 PF possesses fecal protease inhibition, or that it can benefit diapered skin. Therefore, this work shows that OXY 229 PF, and the individual components thereof, can be used to inhibit fecal enzymes and promote healthy diaper skin.

### SUMMARY

OXY229 PF (Saccharomyces Cerevisiae Lysate) possesses the ability to reduce fecal proteases (chymotrypsin and trypsin) activity in a dose-dependent manner in vitro, showing a novel mechanism of action for healthy diaper skin. Based on the work, OXY229 PF (Saccharomyces Cerevisiae Lysate) is a good postbiotic to prevent or reduce fecal protease induced diaper rash.

### TABLES

TABLE 1
Postbiotic candidates for skin health benefit screening

| | | pH@ maximum | | | | |
|---|---|---|---|---|---|---|
| | | recommended | | Recommended | Product | |
| Company | Product | dose | Organisms | dose | Format | INCI name |
| DSM | OXY 229 PF | 4.92 | Saccharomyces | 1-5% | Aqueous | Saccharomyces Lysate, |
| | | | lysate | | solution | Valine, Threonine, |
| | | | | | | Glutamic acid, Glycine, |
| | | | | | | Disodium Succinate |
| BASF | Relipidium ® | 5.54 | Yeast extract | 1-2% | Water | Hydrolyzed Yeast Protein |
| | A00265 | | modified by | | soluble, | (and) Butylene Glycol |
| | | | Lactobacillus | | liquid | (and) Pentylene Glycol |
| Symrise | SymREboot ® | 5.14 | Lactobacillus | 0.1-1% | Powder, | Maltodextrin (and) |
| | L19 | | | | dispersible in | Lactobacillus Ferment |
| | | | | | all solvents | |
| LipoTrue, S.L. | MARSturizer ™ | 5.59 | Bacillus | 1-2% | Water | Propanediol, water, |
| | | | | | soluble, | bacillus ferment |
| | | | | | liquid | |
| LipoTrue, S.L. | Pauseile ™ | 5.90 | Bacillus | 1-2% | Water | Glycerine, water, bacillus |
| | | | | | soluble, | ferment |
| | | | | | liquid | |

TABLE 2
OXY 229 PF Product Information
Product identification
Product code: 50 3345 4 (previous Pentapharm code: 294-02)
Composition

| | | CAS No. | EINECS/ELI |
|---|---|---|---|
| INCI name | Content | NCS | |
| Saccharomyces Lysate | 1-5% | 94350-12-6 | 305-230-8 |
| Valine | <0.1% | 72-18-4 | 200-773-6 |
| Threonine | <0.1% | 72-19-5 | 200-774-1 |
| Glutamic Acid | <0.1% | 56-86-0 | 200-293-7 |
| Glycine | <0.1% | 56-40-6 | 200-272-2 |
| Glycerin | 10-25% | 56-81-5 | 200-289-5 |
| Aqua | >50% | 7732-18-5 | 231-791-2 |
| Disodium Succinate | 0.1-1% | 6106-21-4 | 205-778-7 |
| Phenoxyethanol | 0.1-1% | 122-99-6 | 204-489-7 |
| Ethylhexylglycerin | 0.1-1% | 70445-33-9 | 408-080-2 |

| Specifications | | |
|---|---|---|
| Appearance: | Clear, yellowish solution | |
| pH: | 5.0-6.0 | |
| Water content: | 74.0-80.0% | m/m |
| Relative density d20/20: | 1.050-1.070 | |
| Refractive index n25: | 1.362-1.364 | |
| Nitrogen, total: | 0.06-0.10% | m/m |
| Total aerobic mesophile plate count: | <100 | CFU/ml |

| | |
|---|---|
| Specified microorganisms: | not detectable in one milliliter |
| Solubility | |
| OXY 229 PF is soluble in water | |

### Example 2: Investigation Of Fecal Protease Inhibition By Other Saccharomyces Cerevisiae Lysates

### Background

The benefit of OXY 229 PF formulation from DSM for healthy diapered skin was determined in Example 1. The formulation significantly inhibited fecal protease activity. The result shows that it is useful as a postbiotic candidate that can be incorporated into diaper treatment, wipe or topical formulation to prevent diaper dermatitis. To explore whether the underlying mechanism is due to the general characteristics of Saccharomyces cerevisiae, other S. cerevisiae lysates were tested for protease assay.

### Method

Preparation of Saccharomyces cerevisiae lysate solutions from various vendors

Vitacell®LS 7979 formulation containing S. cerevisiae lysate was obtained from BASF. Another S. cerevisiae extract was purchased from Sigma-Aldrich (CAS #8013 Jan. 2). Fresh OXY 229 PF (2023) and Relipidium formulations were obtained from DSM and from Symrise. The original batch of OXY 229 PF (2021) tested in prior art was also prepared and used as an experimental control. The stock formulations or powder from suppliers were diluted with water (pH 7) to prepare the solution at the maximum recommended dosage according to manufacturers' instructions (2-5%). The pH of the final solutions (2% Vitacell®, 2% Sigma-Aldrich yeast extract. 2% Relipidium, and 5% OXY 229 PF) was measured using a pH meter (Mettler Toledo, SevenExcellence pH meter S400). After pH measurement, the solutions were tested for fecal protease assay (Na-Benzoyl-DL-Arginine-p-Nitroanilide assay) to investigate the impact of various S. cerevisiae lysates formulation on fecal protease activity. Protease assay was conducted as described in Example 1. pH-adjusted water at 7 or 5 was also included in the assay for vehicle control.

### Result and Discussion

Unlike OXY 229 PF, other S. cerevisiae lysates did not inhibit fecal protease activity.

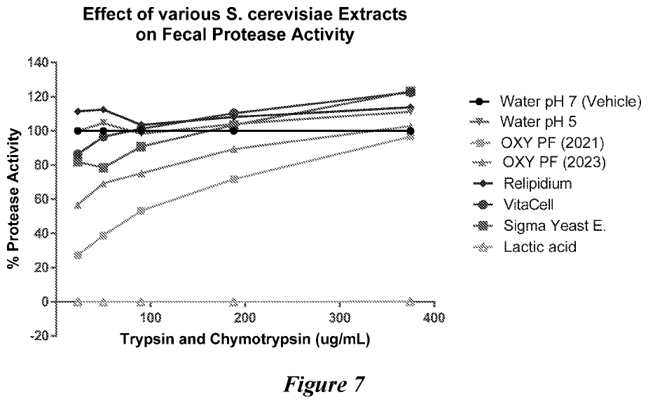

Consistent with the previous observation, OXY 229 PF solution (either 2021 or 2023 batch) reduced fecal enzyme activity compared to vehicle control, whereas Relipidium (Lactobacillus modified yeast extract from Symrise) did not show any inhibitory action (FIG. 7). The protease inhibitory action of OXY 229 PF was consistent and reproducible in multiple rounds of experiment, even by different batches. However, no similar inhibitory actions were observed from other S. cerevisiae lysates from DSM (Vitacell®) or Sigma-Aldrich extract (FIG. 7). This shows that not all S. cerevisiae lysates have protease inhibitory action, rather, strain-specific properties of S. cerevisiae can attribute the effect on fecal protease activity.

Since the pH of OXY 229 PF was lower than others (Table 3), it was determined whether the underlying mechanism is pH driven by comparing the protease activity in the vehicle of the same pH (pH 5). The inhibitory action of protease activity by OXY 229 PF surpassed the level of protease activity found in vehicle prepared at the same pH (pH 5), showing that its action is not mediated by pH, but by other mechanisms yet to be addressed.

Potential compositions modulating fecal protease activity include divalent ions including calcium, magnesium, iron, manganese, zinc, or copper. As mentioned earlier, various metabolites or protease inhibitors produced by specific strains of S. cerevisiae can also contribute to the inhibitory effect observed.

TABLE 3
pH of various S. cerevisiae extracts
and other postbiotic formulation

| Sample | pH |
|---|---|
| 5% OXY 229 PF (2021 batch) | 5.40 |
| 5% OXY 229 PF (2023 batch) | 5.85 |
| 2% Relipidium | 6.30 |
| 2% Vitacell | 7.51 |
| 2% Sigma Yeast Extract | 7.09 |
| 1% lactic acid solution | 2.68 |

Lastly, while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the disclosure. Other implementations of the disclosure will be apparent to those skilled in the art from consideration of the specification and practice of the methods disclosed herein. It is intended that the specification and examples be considered as examples only, with a true scope and spirit of the disclosure being indicated by the following claims.

### EXAMPLE IMPLEMENTATIONS

Example Implementation 1: A method of inhibiting activity of one or more fecal enzymes, the method comprising exposing the fecal enzymes to a postbiotic composition, wherein the postbiotic composition reduces the activity of one or more of the fecal enzymes by 10% or more compared to a control.

Example Implementation 2: The method of example implementation 1, wherein the postbiotic composition comprises Saccharomyces lysate.

Example Implementation 3: The method of example implementation 2, wherein the Saccharomyces lysate is derived from Saccharomyces cerevisiae.

Example Implementation 4: The method of any one of example implementations 1-3, wherein the fecal enzyme is a serine protease.

Example Implementation 5: The method of example implementation 4, wherein the serine protease is chymotrypsin or trypsin.

Example Implementation 6: The method of any one of example implementations 1-5, wherein the postbiotic composition further comprises one or more amino acids.

Example Implementation 7: The method of example implementation 6, wherein the one or more amino acids are selected from the group comprising valine, threonine, glutamic acid, and glycine.

Example Implementation 8: The method of any one of example implementations 1-7, wherein the postbiotic composition further comprises disodium succinate.

Example Implementation 9: The method of any one of example implementations 1-8, wherein the postbiotic composition comprises OXY 229 PF.

Example Implementation 10: The method of example implementation 9, wherein the amount of OXY 229 PF is between 0.5% to 10%.

Example Implementation 11: The method of any one of example implementations 1-9, wherein said fecal enzymes occur on an epidermal surface.

Example Implementation 12: The method of any one of example implementations 1-10, wherein the fecal enzyme activity is reduced by 10-90% or more compared to a control.

Example Implementation 13: A method of preventing or reducing fecal enzyme-related diaper dermatitis in a subject in need thereof, the method comprising providing a postbiotic composition to the subject, wherein the postbiotic composition helps reduce or prevent fecal enzyme-related diaper dermatitis by 1% or more compared to a control.

Example Implementation 14: The method of example implementation 13, wherein the fecal-enzyme-related dermatitis is caused by one or more fecal enzymes.

Example Implementation 15: The method example implementation 14, wherein said one or more fecal enzymes occur on an epidermal surface.

Example Implementation 16: The method of example implementation 14, wherein the fecal enzyme is a serine protease.

Example Implementation 17: The method of example implementation 16, wherein the serine protease is chymotrypsin or trypsin.

Example Implementation 18: The method of any one of example implementations 13-17, wherein the postbiotic composition comprises Saccharomyces lysate.

Example Implementation 19: The method of example implementation 17, wherein the Saccharomyces lysate is derived from Saccharomyces cerevisiae.

Example Implementation 20: The method of any one of example implementations 13-19, wherein the postbiotic composition further comprises one or more amino acids.

Example Implementation 21: The method of example implementation 20, wherein the one or more amino acids are selected from the group comprising valine, threonine, glutamic acid, and glycine.

Example Implementation 22: The method of any one of example implementations 13-22, wherein the postbiotic composition further comprises disodium succinate.

Example Implementation 23: The method of any one of example implementations 13-22, wherein the postbiotic composition comprises OXY 229 PF.

Example Implementation 24: The method of example implementation 23, wherein the amount of OXY 229 PF is between 0.5% and 10%.

Example Implementation 25: The method of any one of example implementations 13-24, wherein protease-induced diaper dermatitis is reduced by 5% or more.

Example Implementation 26: An absorbent article comprising a composition, wherein the composition comprises Saccharomyces lysate.

Example Implementation 27: The absorbent article of example implementation 26, wherein the composition further comprises one or more amino acids.

Example Implementation 28: The absorbent article of example implementation 27, wherein the one or more amino acids are selected from the group comprising valine. threonine, glutamic acid, and glycine.

Example Implementation 29: The absorbent article of any one of example implementations 26-28, wherein the composition further comprises disodium succinate.

Example Implementation 30: The absorbent article of any one of example implementations 26-29, wherein the Saccharomyces lysate is derived from Saccharomyces cerevisiae.

Example Implementation 31: The absorbent article of any one of example implementations 26-30, wherein the composition is OXY 229 PF.

Example Implementation 32: The absorbent article of example implementation 29, wherein OXY 229 PF is present at a concentration of 0.5% to 10%.

Example Implementation 33: The absorbent article of any one of example implementations 26-32, wherein the absorbent article comprises diapers, diaper pants, training pants, youth pants, swim pants, feminine hygiene products, adult care garments, medical garments, surgical pads and bandages, other personal care or health care garments.

Example Implementation 34: The absorbent article of example implementation 33, wherein the composition is disposed on a side of the absorbent article configured for contact with a wearer.

Example Implementation 35: The absorbent article of example implementation 34, wherein the side of the absorbent article configured for contact with a wearer comprises a bodyside liner.

1. A method of inhibiting activity of one or more fecal enzymes, the method comprising exposing the fecal enzymes to a postbiotic composition, wherein the postbiotic composition reduces the activity of one or more of the fecal enzymes by 10% or more compared to a control.

2. The method of claim 1, wherein the postbiotic composition comprises Saccharomyces lysate.

3. The method of claim 2, wherein the Saccharomyces lysate is derived from Saccharomyces cerevisiae.

4. The method of claim 1 wherein the fecal enzyme is a serine protease.

5. The method of claim 4, wherein the serine protease is chymotrypsin or trypsin.

6. The method of claim 1, wherein the postbiotic composition further comprises one or more amino acids.

7. The method of claim 6, wherein the one or more amino acids are selected from the group comprising valine, threonine, glutamic acid, and glycine.

8. The method of claim 1, wherein the postbiotic composition further comprises disodium succinate.

9. The method of claim 1, wherein the postbiotic composition comprises OXY 229 PF.

10. The method of claim 9, wherein the amount of OXY 229 PF is between 0.5% to 10%.

11. The method of claim 1, wherein said fecal enzymes occur on an epidermal surface.

12. The method of claim 1, wherein the fecal enzyme activity is reduced by 10-90% or more compared to a control.

13. A method of preventing or reducing fecal enzyme-related diaper dermatitis in a subject in need thereof, the method comprising providing a postbiotic composition to the subject, wherein the postbiotic composition helps reduce or prevent fecal enzyme-related diaper dermatitis by 1% or more compared to a control.

14. The method of claim 13, wherein the fecal-enzyme-related dermatitis is caused by one or more fecal enzymes.

15. The method claim 14, wherein said one or more fecal enzymes occur on an epidermal surface.

16-25. (canceled)

26. An absorbent article comprising a composition, wherein the composition comprises Saccharomyces lysate.

27-32. (canceled)

33. The absorbent article of claim 26, wherein the absorbent article comprises diapers, diaper pants, training pants, youth pants, swim pants, feminine hygiene products, adult care garments, medical garments, surgical pads and bandages, other personal care or health care garments.

34. The absorbent article of claim 33, wherein the composition is disposed on a side of the absorbent article configured for contact with a wearer.

35. The absorbent article of claim 34, wherein the side of the absorbent article configured for contact with a wearer comprises a bodyside liner.
